(19) United States
(12) Patent Application Publication
(10) Pub. No.: US 2011/0201084 A1
(43) Pub. Date: Aug. 18, 2011

(54) ENZYMATIC HYDROLYSIS OF CELLULOSIC BIOMASS THROUGH ENHANCED REMOVAL OF OLIGOMERS

(75) Inventors: Charles E. Wyman, Riverside, CA (US); Rajeev Kumar, Riverside, CA (US)

(73) Assignee: THE REGENTS OF THE UNIVERSITY OF CALIFORNIA, Oakland, CA (US)

(21) Appl. No.: 13/055,366

(22) PCT Filed: Jul. 24, 2009
(86) PCT No.: PCT/US09/51737

§ 371 Date: May 9, 2011

Related U.S. Application Data
(60) Provisional application No. 61/083,826, filed on Jul. 25, 2008.
(60) Provisional application No. 61/144,118, filed on Jan. 12, 2009.

(51) Int. Cl. C12N 9/42; C02F 3/34; C12M 1/00

(52) U.S. Cl. 435/209; 435/262; 435/289.1

(57) ABSTRACT

The methods and composition of the disclosure are directed to the reducing cellulase inhibition by xylooligomers through adding hemicellulase enzymes that break xylooligomers down to monomeric sugars with a corresponding increase in glucose release. What was heretofore unknown is that xylobiose and higher xylooligomers inhibit enzymatic hydrolysis of any lignocellulosic biomass (plant material) used as feed stocks for commercial ethanol production, including, but not limited to, corn stover, birch wood, switchgrass, poplar wood, Miscanthus, aspen, other grasses and woody plants, and many other types of agricultural and forestry residues, as well as municipal solid waste. In addition, the examples herein show that xylobiose and xylotriose have progressively greater effects on hydrolysis rates. In accordance with the disclosure, the addition of one or more hemicellulase enzymes, such as xylanase and β-xylosidase, to a lignocellulosic biomass significantly improves hydrolysis performance.

### CROSS REFERENCE TO RELATED APPLICATIONS

This application claims priority to U.S. Provisional Application No. 61/144,118, filed Jan. 12, 2009 and U.S. Provisional Application No. 61/083,826, filed Jul. 25, 2008, the disclosures of which are incorporated herein by reference in their entirety.

### FIELD OF THE INVENTION

The disclosure is directed to methods and systems for removing carbohydrate oligomers formed during pretreatment and enzymatic hydrolysis of lignocellulosic biomass to increase performance of sugar release, and more particularly to the addition of one or more hemicellulase enzymes that breakdown oligomers prior to and/or during enzymatic hydrolysis.

### BACKGROUND

The U.S. transportation sector is the largest contributor to greenhouse gas (GHG) emissions and is almost totally fueled by petroleum, about two thirds of which is imported with unstable regions holding most reserves. Conversion of inexpensive and abundant cellulosic biomass to fuels has powerful attributes for production of sustainable transportation fuels. Biological conversion of cellulosic biomass to ethanol is particularly promising as a low cost route to reducing GHG emissions and producing sustainable transportation fuels due to the very high yields possible, the dramatic cost reductions that have been realized, and the power of modem biotechnology for substantial additional cost reductions.

What is needed is lower cost technologies for the integrated operations of pretreatment, enzymatic hydrolysis, and fermentation that dominate conversion costs to ethanol for existing biomass sources selected for their abundance, low cost, and susceptibility. In particular, advanced technologies need to be developed to enhance ethanol yields while reducing costs. Such technologies would provide a foundation for industry to commercialize cellulosic ethanol technology.

Cellulosic ethanol offers powerful benefits including near zero net greenhouse gas emissions, enhanced energy security and balance of trade, and creation of rural jobs. To support process development, valuable data should be assembled in collaboration with experts on the availability, composition, and cost of biomass that is promising for ethanol production. The resulting feedstock and process information will facilitate assessments by industry of opportunities to competitively make ethanol from major fractions of agricultural residues and municipal solid waste and accelerate commercialization of processes for converting low cost cellulosic biomass into valuable products with major societal benefits.

### SUMMARY

The disclosure is directed to methods and systems for enhancing performance of cellulase enzymes and/or enabling the effective use of lower amounts of cellulase enzymes for hydrolysis of lignocellulosic biomass to release sugars for fermentation to ethanol and other products. A mixture of enzyme activities known as cellulase work collectively to break down cellulose in biomass to release glucose. Cellulase activity is any enzymatic hydrolysis of 1,4-beta-D-glycosidic linkages in cellulose by endoglucanases, cellobiohydrolases, and other enzymes. Cellulase activity can be inhibited by various sugar oligomers that are produced by the hydrolysis of cellulose and hemicelluloses in the reaction mixture. The disclosure is based on the addition of one or more enzymes that degrade xylose oligomers before or during enzymatic hydrolysis of lignocellulosic biomass so as to reduce inhibition of cellulase enzyme by xylose oligomers released during pretreatment and/or enzymatic hydrolysis. In one embodiment, the methods, bioreactors, systems and compositions of the disclosure use xylose oligomer degrading enzymes that degrade oligomers of 3-8 (e.g., 3, 4, 5, 6, 7, or 8) sugars in length.

Glucose and particularly cellobiose are known to be powerful inhibitors of cellulase enzymes. It has been known that supplementing cellulase with an enzyme known as beta-glucosidase (β-glucosidase) accelerates the conversion of the cellobiose produced to glucose and improves sugar yields. One aspect of the disclosure is the discovery that that oligomers of xylose are also very strong inhibitors of cellulase enzymes. Thus, the disclosure employs supplementation of cellulase with xyolose oligomer degrading enzymes, such as xylanase and beta-xylosidase (β-xylosidase) that break down these oligomers to monomers, such as xylose, that are less inhibitory. In accordance with the disclosure, supplementation with xylanase and β-xylosidase reduces the inhibition of cellulase enzymatic hydrolysis by xylose oligomers and oligomers of other sugars formed during breakdown of hemicelluloses, providing an increase in glucose and xylose release, thereby reducing cellulase enzyme demand for ethanol production or enhancing performance for existing loadings. Similarly, xylanas and xylose oligomer degrading enzyme supplementation in accordance with the disclosure reduces the inhibition of enzymatic hydrolysis by xylobiose and higher xylooligomers.

In one embodiment of the disclosure, moderate loadings of cellulase enzyme supplemented with β-xylosidase are applied to solids produced by known or yet to be developed pretreatment methods. Presently known pretreatment processes for lignocellulosic biomass include ammonia fiber expansion (AFEX), ammonia recycled percolation (ARP), and pretreatment with dilute sulfuric acid, with lime, with sulfur dioxide or processing in a neutral pH environment. Supplementation of enzymatic digestion of the solids from such lignocellulosic biomass pretreatment methods with β-xylosidase and/or xylanase provides higher glucose removal with lower cellulase enzyme loadings than are achieved from using the same amounts of cellulase enzymes alone.

In another aspect of the invention, β-xylosidase and/or xylase are included in the reaction mixture simultaneously with hydrolysis by cellulases. In yet another aspect of the invention, xylanases are removed from pretreated biomass or are removed during hydrolysis by cellulase enzymes by non-enzymatic means.

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 depicts equivalent glucose yields at various β-glucosidase loadings for different pretreatment methods.

FIG. 2 depicts xylose yields at various β-glucosidase loadings for different pretreatment methods.

FIG. 3a depicts the effect of deacetylation on glucan and xylan removal.

FIG. 3b depicts glucose yield compared to xylose yield for untreated and deacetylated corn stover.

FIG. 4 depicts the effect of different pretreatment methods on the post hydrolysis yields of cellobiose, of higher cellooligomers and of xylooligomers.

FIGS. 5a and 5b depicts the effect on glucose yield over time by the addition of xylan, xylose, and β-xylosidase.

FIGS. 6a-6d depicts the effect of β-glucosidase supplementation at a fixed cellulase mass loading on glucose release from amorphous cellulose, highly crystalline cellulose, and cellulose in corn stover solids left after SO₂ and dilute sulfuric acid pretreatment.

FIG. 7 depicts the effect of β-xylosidase supplementation on xylose release from birch wood xylan at fixed xylanase mass loadings of (a) seven mg protein/g xylan and (b) fourteen mg protein/g xylan.

FIGS. 8a and 8b depict the effect of β-xylosidase supplementation on glucose, xylose, and xylooligomer release at a fixed cellulase plus β-glucosidase mass loading of 16.1 mg/g original glucan for AFEX pretreated corn stover and SO₂ pretreated corn stover

FIG. 9 depicts the effect of xylose oligomer chain length on Avicel glucan initial hydrolysis rate at a cellulase loading of thirty FPU/g glucan and CBU to FPU activity ration of two.

FIG. 10 shows glucose and xylose yields after 72 h of hydrolysis vs. total protein loadings for xylanase supplementation of constant cellulase loadings corresponding to about 14.5 and 29 mg protein/g original glucan for AFEX pretreated corn stover solids. Closed symbols represent glucose yields and open symbols designate xylose yields. A and B are glucose and xylose yields at 14.5 mg cellulase+MX5+b-xylosidase. Error bars represent the standard deviation.

FIG. 11 shows glucose and xylose yields after 72 h of hydrolysis vs. total protein loadings for xylanase supplementation of constant cellulase loadings corresponding to about 14.5 and 29 mg protein/g original glucan for ARP pretreated corn stover solids. Closed symbols represent glucose yields and open symbols designate xylose yields. Error bars represent the standard deviation.

FIG. 12 shows glucose and xylose yields after 72 h of hydrolysis vs. total protein loadings for xylanase supplementation of constant cellulase loadings corresponding to about 14.5 and 29 mg protein/g original glucan for dilute acid pretreated corn stover solids. Closed symbols represent glucose yields and open symbols designate xylose yields. A and B are glucose and xylose yields at 14.5 mg cellulase+b-xylosidase.

FIG. 13 shows glucose and xylose yields after 72 h of hydrolysis vs. total protein loadings for xylanase supplementation of constant cellulase loadings corresponding to about 14.5 and 29 mg protein/g original glucan for lime pretreated corn stover solids. Closed symbols represent glucose yields and open symbols designate xylose yields. Error bars represent the standard deviation.

FIG. 14 shows glucose and xylose yields after 72 h of hydrolysis vs. total protein loadings for xylanase supplementation of constant cellulase loadings corresponding to about 14.5 and 29 mg protein/g original glucan for controlled pH pretreated corn stover solids. Closed symbols represent glucose yields and open symbols designate xylose yields. Error bars represent the standard deviation.

FIG. 15 shows glucose and xylose yields after 72 h of hydrolysis vs. total protein loadings for xylanase supplementation of constant cellulase loadings corresponding to about 14.5 and 29 mg protein/g original glucan for SO₂ pretreated corn stover solids. Closed symbols represent glucose yields and open symbols designate xylose yields. A and B are glucose and xylose yields at 14.5 mg cellulase+b-xylosidase.

FIG. 16A-B shows relationship between release of glucose and removal of xylose as a result of enzymatic hydrolysis with fixed cellulase loadings of 14.5 and 29.0 mg/g original glucan and different levels of xylanase supplementation for corn stover solids produced by (a) AFEX and (b) ARP pretreatments.

FIG. 17A-C shows (a) Glucose and xylose yields after 72 h of hydrolysis for xylanase supplementation of constant cellulase mass loadings of 29 mg/g original glucan for untreated (UT CS) and deacetylated (54%-De-B, 100%-De-D) corn stover solids; (b) changes in glucan conversion with xylan conversion for UT and De CS; and (c) impact of deacetylation of biomass on cellobiose generation for hydrolysis with purified CBH-I. Error bars represent the standard deviation.

FIG. 18 shows predicted vs. experimental xylanase leverage for two CTB loadings for xylanase supplementation mass ratios up to 11.

FIG. 19 shows the effect of xylooligomers on the 4 h rate of 1% (w/v) Avicel glucan digestion at a cellulase loading of 7.5 FPU/g glucan. Avicel+xylan−1% (w/v) each, Avicel+xylose−xylose equivalent to 1% (w/v) xylan. b-xylosidase loading was 28 mg/g glucan. Error bars represent the standard deviation.

### DETAILED DESCRIPTION

As used herein and in the appended claims, the singular forms “a,” “and,” and “the” include plural referents unless the context clearly dictates otherwise. Thus, for example, reference to “an enzyme” includes a plurality of such enzymes and reference to “the material” includes reference to one or more materials known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Cellulosic and lignocellulosic feedstocks and wastes, such as agricultural residues, wood, forestry wastes, sludge from paper manufacture, and municipal and industrial solid wastes, provide a potentially large renewable feedstock for the production of chemicals, plastics, fuels and feeds. Cellulosic and lignocellulosic feedstocks and wastes, composed of carbohydrate polymers comprising cellulose, hemicellulose, and lignin can be generally treated by a variety of chemical, mechanical and enzymatic means to release primarily hexose and pentose sugars, which can then be fermented to useful products including ethanol.

For biomass, pretreatments by ammonia fiber expansion (AFEX), ammonia recycle percolation (ARP), controlled (neutral) pH, dilute acid (DA), and lime processes were previously shown to achieve similarly high glucose and xylose yields for the combined operations of pretreatment and enzymatic hydrolysis at high cellulase mass loadings of about fifty-five and two-hundred twenty mg of protein/g glucan in the original corn stover (corresponding to about fifteen FPU and sixty FPU/g glucan, respectively). Furthermore, the majority of results available in the literature for pretreated solids report high saccharification yields using uneconomically high enzyme loadings. However, because lower enzyme loadings that still achieve high sugar yields are essential to economic success, better knowledge is needed of factors that govern sugar release at more moderate enzyme loadings. Nonproductive binding of cellulase and other enzymes with lignin and other portions of the solid, inhibition by sugar and their degradation compounds, and their inactivation over time are believed by those having ordinary skill in the art to be at least partly responsible for high enzyme loading requirements, but the complexity of the substrate and enzymes has confounded developing a clear picture of the mechanism. Heretofore, it has not been determined whether carbohydrate oligomers other than cellobiose inhibit cellulase action because these higher oligomers are almost nonexistent at the higher enzyme loadings typically used.

The methods of the disclosure provide enhanced sugar release patterns for biomass solids prepared by different pretreatments when subjected to hemicellulase enzyme loadings. In the examples herein, glucose and xylose release from corn stover solids pretreated by leading technologies of ammonia fiber expansion (AFEX), ammonia recycled percolation (ARP), dilute sulfuric acid (DA), lime; controlled (neutral) pH (NpH), and sulfur dioxide (SO₂) as well as pure cellulose and xylan were followed for up to seventy-two hours of hydrolysis over a range of cellulase enzyme loadings. In addition, the effect of corn stover deacetylation on twenty-four hour release of glucose and xylose was determined. One embodiment of the disclosure is directed to the impact of xylooligomers on sugar release and the benefits of supplementation with xylanase and β-xylosidase enzymes.

The methods of the disclosure further include the use of xylose oligomer degrading enzymes in the hydrolysis reaction mixture, simultaneous to the exposure of biomass to cellulases. The methods of the disclosure can alternatively or further include the removal of xylose oligomers from pretreated biomass or simultaneous with cellulase treatment by non-enzymatic means. Such means include any physical, chemical, or other method which acts to remove, degrade, sequester, or otherwise reduce the levels of xylose oligomers in biomass mixtures. Such means include but are not limited to acid pretreatment, removal by filtration, removal by antibodies, dialysis, fractionation, precipitation and others.

Cellulases refer to a generic class of enzymes comprising exo-cellobiohydrolases (CBH), endoglucanases (EG), β-glucosidase, xylanases and β-xylosidase. The CBH and EG enzymes catalyze the hydrolysis of the cellulose (β-1,4-D-glucan linkages). The CBH enzymes, CBHI and CBHII, act on the ends of the glucose polymers in cellulose microfibrils and liberate cellobiose, while the EG enzymes act at random locations on the cellulose. Together, cellulase enzymes hydrolyze cellulose to cellobiose, which, in turn, is hydrolyzed to glucose by β-glucosidase. The xylanase enzymes, such as xylanase 1 (Xyn1), xylanase 2 (Xyn2) and β-xylosidase, are typically present in the cellulase enzyme mixture and hydrolyze any xylan present in the feedstock.

Cellulolytic activity: The term “cellulolytic activity” is defined herein as a biological activity that hydrolyzes a cellulose-containing material. Cellulolytic protein may hydrolyze microcrystalline celluose or other cellulosic substances, thereby decreasing the mass of insoluble cellulose and increasing the amount of soluble sugars. The reaction can be measured by the detection of reducing sugars with p-hydroxybenzoic acid hydrazide, a high-performance-liquid-chromatography (HPLC), or an electrochemical sugar detector. Determination of cellulase activity, measured in terms of Cellulase Viscosity Unit (CEVU), quantifies the amount of catalytic activity present in a sample by measuring the ability of the sample to reduce the viscosity of a solution of carboxymethyl cellulose (CMC). The assay is performed at a temperature and pH suitable for the cellulolytic protein and substrate.

In one embodiment, cellulolytic activity is determined by measuring the increase in hydrolysis of a cellulosic material by a cellulolytic enzyme composition under the following conditions: 1-10 mg of cellulolytic protein/g of cellulose in PCS for 5-7 days at 50° C. compared to a control hydrolysis without addition of cellulolytic protein.

Endoglucanase: The term “endoglucanase” is defined herein as an endo-1,4-(1,3; 1,4)-beta-D-glucan 4-glucanohydrolase (E.C. No. 3.2.1.4), which catalyses endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. For purposes of the present invention, endoglucanase activity is determined using carboxymethyl cellulose (CMC) hydrolysis according to the procedure of Ghose, 1987, Pure and Appl. Chem. 59: 257-268.

Beta-glucosidase: The term “beta-glucosidase” is defined herein as a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the procedure described by Venturi et al., 2002, J. Basic Microbiol. 42: 55-66. One unit of beta-glucosidase activity is defined as 1.0 μmole of p-nitrophenol produced per minute at 50° C., pH 5 from 4 mM p-nitrophenyl-beta-D-glucopyranoside as substrate in 100 mM sodium citrate, 0.01% TWEEN®20.

Cellulosic material: The predominant polysaccharide in the primary cell wall of biomass is cellulose, the second most abundant is hemi-cellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

The cellulosic material can be any material containing cellulose. Cellulose is generally found, for example, in the stems, leaves, hulls, husks, and cobs of plants or leaves, branches, and wood of trees. The cellulosic material can be, but is not limited to, herbaceous material, agricultural residue, forestry residue, municipal solid waste, waste paper, and pulp and paper mill residue. The cellulosic material can be any type of biomass including, but not limited to, wood resources, municipal solid waste, wastepaper, crops, and crop residues (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix.

In one embodiment, the cellulosic material is herbaceous material. In another embodiment, the cellulosic material is agricultural residue. In another embodiment, the cellulosic material is forestry residue. In another embodiment, the cellulosic material is municipal solid waste. In another embodiment, the cellulosic material is waste paper. In another embodiment, the cellulosic material is pulp and paper mill residue.

In another embodiment, the cellulosic material is corn stover. In another embodiment, the cellulosic material is corn fiber. In another embodiment, the cellulosic material is corn cob. In another embodiment, the cellulosic material is orange peel. In another embodiment, the cellulosic material is rice straw. In another embodiment, the cellulosic material is wheat straw. In another embodiment, the cellulosic material is switch grass. In another embodiment, the cellulosic material is miscanthus. In another embodiment, the cellulosic material is bagasse.

Pretreatment methods are used to make the carbohydrate polymers of cellulosic and lignocellulosic materials more readily available to saccharification enzymes. Standard pretreatment methods have historically utilized primarily strong acids at high temperatures; however due to high energy costs, high equipment costs, high pretreatment catalyst recovery costs and incompatibility with saccharification enzymes, alternative methods are being developed, such as enzymatic pretreatment, or the use of acid or base at milder temperatures where decreased hydrolysis of biomass carbohydrate polymers occurs during pretreatment, requiring improved enzyme systems to saccharify both cellulose and hemicellulose.

For example, a pretreated biomass slurry is neutralized if necessary, as in the case of dilute acid pretreatment, using a base (e.g. NaOH), and then a buffer and appropriate nutrients are added to the slurry to reach the same final concentration as for the separated and washed solids (0.05M). This slurry can be spiked with enzymes to break down the polymeric carbohydrate fractions in the pretreated biomass. The method is referred to as “co-hydrolysis” because the hydrolysis is done with the liquid released in pretreatment being present. The enzyme mixture for co-hydrolysis may be enriched with xylanase when a considerable amount of the xylan fraction may be left in the pretreated solids. Additionally the concentration of soluble xylooligomers could reach enzyme inhibitory levels and may need to be reduced. Xylanases and/or beta-xylosidases break down these oligomers in solution to monomeric xylose, which is not enzyme inhibiting.

Xylose and xylooligomers inhibit the activity of cellulases and thus inhibit the efficiency of biomass hydrolysis. For example, the disclosure demonstrates that xylobiose and xylotriose have progressively greater inhibitory effects on hydrolysis rates and that their removal promotes sugar production and cellulase activity. The examples provided herein demonstrate that xylobiose and higher xylooligomers inhibit enzymatic hydrolysis of pure glucan, pure xylan, and pretreated biomass such as corn stover.

The methods and composition of the disclosure are directed to the reducing cellulase inhibition by xylooligomers through adding xylose and xylose oligomer degrading enzymes that break xylooligomers down to monomeric sugars with a corresponding increase in glucose release. In accordance with the disclosure, the addition of one or more enzymes that degrade xylose and xylose oligomers, such as xylanase and β-xylosidase, to a lignocellulosic biomass significantly improves hydrolysis performance.

The methods, systems and compositions of the disclosure are applicable to any pretreated lignocellulosic biomass used as feed stocks for commercial ethanol production, including, but not limited to, corn stover, birch wood, switchgrass, poplar wood, Miscanthus grass, aspen, and other grasses woody plants and many other types of agricultural and forestry residues, as well as municipal solid waste.

As used herein a xylan, xylose, or xylose oligomer degrading enzyme refers to a polypeptide that is capable of catalyzing the degradation of a xylan, xylose or oligomer thereof to smaller subunits or to glucose or other sugars. The enzyme may be used in conjunction with chemical and heat treatments to promote degradation of a xylose or xylose oligomers.

Enzymes useful for xylose and xylose oligomer degradation include, but are not limited to, xylanase and β-xylosidase. In one embodiment, a xylanase can be used to degrade oligomers of xylose. The xylanase may be of microbial origin, such as fungal origin (e.g., Trichoderma, Meripilus, Humicola, Aspergillus, Fusarium) or bacterial origin (e.g., Bacillus). In a one embodiment, the xylanase is obtained from a filamentous fungus, preferably from a strain of Aspergillus, such as Aspergillus aculeatus; or a strain of Humicola, such as Humicola lanuginosa. The xylanase is preferably an endo-1,4-beta-xylanase, more commonly an endo-1,4-beta-xylanase of GH10 or GH11. Examples of commercial xylanases include Shearzyme™ (Novozymes A/S, Denmark).

It will be recognized that engineered, variants and modified enzymes having xylose and xylose oligomer degradation activity can be used in the methods of the disclosure. Such modified enzymes may comprise increased activity compared to a wild-type or increase thermo- or solvent stability (see, e.g., U.S. Patent Publication No. 20090155238, which described thermostable xylanases, the disclosure of which is incorporated herein). Methods of determining xylanase activity are known in the art. For example, “xylanase activity” is defined herein as a 1,4-beta-D-xylan-xylanohydrolase activity (E.C. 3.2.1.8) that catalyzes the endohydrolysis of 1,4-beta-D-xylosidic linkages in xylans. For purposes of the disclosure, xylanase activity can be determined using 0.2% AZCL-arabinoxylan as substrate in 0.01% Triton X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6 buffer. Thus, the type or source of a xylose or xylose oligomer degrading enzyme is not limited by the disclosure. Identification of various enzymes having xylose degrading or xylose-oligomer degrading activity can be routinely performed in the art. Furthermore, modified enzymes having xylose/xylose-oligomer degrading activity can be used and include those having increase stability, thermo-stability, solvent stability and/or increased activity. Again, various sources of polypeptides having xylanase activity are available to the skilled artisan. A search of an available database identified over 9000 accession numbers describing polypeptides having xylanase activity. Exemplary polypeptides having xylanase activity include, but are not limited to (the sequence of which are incorporated herein): xylanase [Filobasidium floriforme]-gi|295924|emb|CAA31109.1|[295924]; xylanase [Prevotella ruminicola]-gi|143974|gb|AAC36862.1|[143974]; xylanase [Setosphaeria turcica]-gi|5725372|emb|CAB52417.1|[5725372]; xylanase [Streptomyces sp. EC3]-gi|531768|emb|CAA56935.1|[531768]; xylanase [Phacdon cochleariae]-gi|4210810|emb|CAA76932.1|[4210810]; xylanase [synthetic construct]-gi|57231730|gb|AAW47578.1|[57231730]; xylanase [Rhodopirellula baltica SH 1]-gi|32476421|ref|NP_{—}869415.1|[32476421]; xylanase [Streptomyces coelicolor A3(2)]-gi|31340551|ref|NP_{—}733550.1|[31340551]; xylanase [Geobacillus stearothermophilus]-gi1499715|dbj|BAA05669.1|[499715]; xylanase [Aspergillus niger]-gi|225200531|gb|ACN82438.1|[225200531]; xylanase [Paenibacillus sp. BPL-001]-gi|210076633|gb|ACJ06666.1|[210076633]; xylanase [uncultured organism]-gi|57639627|gb|AAW55667.1|[57639627]; xylanase [Clostridium stercorarium]-gi|984792|dbj|BAA02584.1|[984792]; xylanase [Geobacillus stearothermophilus]-gi|73332107|gb|AAZ74783.1|[73332107]; xylanase [Bacillus subtilis]-gi|607050|emb|CAA84276.1|[607050]; and xylanase; beta(1,3-1,4)-glucanase [Ruminococcus flavefaciens]-gi|385911|gb|AAB26620.1∥bbm|302498|bbs|131872[385911].

Exemplary enzymes for use in the methods of the disclosure include β-xylosidase from Trichoderma reesei, as described in “The β-xylosidase of Trichoderma reesei is a multifunctional β-D-xylan xylohydrolase” by Herrmann et al., J Biochem 321:375 (1997), incorporated herein by reference, or the β-xylosidase from Taloromyces emersonii as described in “Mode of action and properties of β-xylosidase from Taloromyces emersonii and Trichoderma reesei,” by Rassmussen et al., Biotechnology and Bioengineering 94:869 (2006), incorporated herein by reference.

The disclosure includes a method for digesting a pretreated lignocellulosic biomass that comprises treating the pretreated lignocellulosic biomass with one or more hemicellulase enzymes that break down xylooligomers to xylose. One embodiment of the method of the disclosure includes supplementing a cellulase preparation with xylanase and/or β-xylosidase (or other xylose or xylose-oligomer degrading enzyme) for enzymatic hydrolysis of the pretreated solids. Another embodiment of the disclosure includes adding xylanase and/or β-xylosidase (or other xylose or xylose-oligomer degrading enzyme) to the liquid fraction resulting from a lignocellulosic biomass pretreatment process, such as, but not limited to, ammonia fiber expansion (AFEX), ammonia recycled peculation (ARP), adding dilute sulfuric acid, adding lime, processing under neutral pH conditions, and treatment with sulfur dioxide. One can also employ the xylose oligomer degrading enzymes with the combined liquid and solids from pretreatment to reduce concentrations of inhibitory xylooligomers in the liquid from pretreatment as well as to react those released into solution by enzyme action on the pretreated solids.

### EXAMPLES

In one example of demonstrating the disclosure, pure cellulose, Avicel PH-101, was obtained from FMC Corporation, Philadelphia, Pa. (Cat 11365, Lot 1094627), and birch wood and beech wood xylans were obtained from Sigma Chemicals, St. Louis, Mo. Amorphous cellulose was prepared from Avicel PH 101 cellulose using concentrated phosphoric acid. Xylobiose and xylotriose were obtained from Megazyme International Ireland, Limited (Bray Business Park, Bray, Co. Wicklow, Republic of Ireland). Unpretreated corn stover was provided by the National Renewable Energy Laboratory (NREL) in Golden, Colo. from a source at Kramer Farm in Colorado. Solids resulting from corn stover pretreatment by various technologies were obtained from the Biomass Refining Consortium for Applied Fundamentals and Innovation (CAFI): ARP by Auburn University, AFEX by Michigan State University, dilute acid pretreatment with the Sunds pilot reactor by NREL, neutral pH by Purdue University, lime by Texas A&M University, and sulfur dioxide by the University of British Columbia. Corn stover was also pretreated using dilute sulfuric acid in a one liter Parr reactor. Reaction conditions and solids compositions as measured according to NREL Laboratory Analytical Procedure 02 (NREL, 2004) are reported in Table 1 for all of the pretreatments. Corn stover was deacetylated with potassium hydroxide (KOH) at room temperature with a total solids concentration of five percent (w/w) on a dry basis. Pretreatment conditions and the composition of the resulting solids as determined by NREL Laboratory Analytical Procedure 002 (NREL, 2004) are reported in Table 1.

TABLE 1
Pretreatment methods, conditions, percent of glucose and xylose left in solids,
and solids compositions for solids prepared by leading technologies.

| | | | Percent of original left in | | Composition of pretreated | | |
|---|---|---|---|---|---|---|---|
| | | | pretreated solids (%) | | solids (%) | | |
| Pretreatment | Pretreatment conditions | | Glucan | Xylan | Glucan | Xylan | Lignin |
| AFEX | 90° C., 220 psi, 1:1 NH₃ to Biomass, 5 min- NW | | 100.0 | 100.0 | 34.4 | 22.8 | 18.0 |
| ARP | 170° C., 525 psi, 3.33:1 NH3; Dry Wt, 20 min, and | | 98.6 | 48.1 | 61.9 | 17.9 | 8.8 |
| | 3.3 ml/g of corn stover - W | | | | | | |
| Dilute acid | Sunds | 180° C., 0.03H₂SO₄; Dry wt, 90 s, 25% | 93.4 | 27.2 | 59.3 | 9.3 | 22.5 |
| | System | solids-HW | | | | | |
| | Parr | 160° C., 0.005 H₂SO₄; Dry wt, 20 min, | 94.0 | NA | 64.4 | 2.9 | 26.4 |
| | Reactor | 5% solids-NW | | | | | |

| | | | | | | |
|---|---|---|---|---|---|---|
| Lime | 55° C., 0.5:1 Ca(OH)₂ to Biomass (dry wt), 4 weeks, | 97.1 | NA | 56.7 | 26.4 | 14.6 |
| | water loading- 10 gm/gm dry biomass- W1 | | | | | |
| Neutral pH | 190° C., 15 min(+5 min heat up)- NW | 94.1 | NA | 52.7 | 16.2 | 25.2 |
| SO₂ | 190° C., 3 min, 3% SO₂- steam explosion-W | 96.9 | NA | 56.9 | 11.6 | 23.8 |
| Diacetylation* | 25° C. 48 h, 1.5 Mmol KOH/gm corn stover(dry wt.)- W | — | — | 42.1 | 23.4 | 19.5 |

NW—not washed, W—water washed, HW—hot water washed, W1—neutralized and washed
*Complete deacetylated corn stover. The untreated corn stover contained 38.3 ± 2.2% glucan, 21.7 ± 1.2% xylan, and 20.5 ± 1.1% lignin.
NA—not available

Spezyme® CP cellulase (lot 301-04075-034; 59±5 FPU/ml, 123±10 mg protein/ml), GC® 220 cellulase (lot 301-04232-162; 90±5 FPU/ml, 184±10 mg protein/ml), Multifect®Xylanase (lot 301-04021-015; 42±5 mg protein/ml), β-glucosidase (31±5 mg protein/ml), and β-xylosidase (75±5 mg protein/ml) enzymes were obtained from Genencor Division of Danisco US, Inc. (Rochester, N.Y., USA). β-glucosidase (Novozyme 188, 140±5 mg protein/ml; 665 CBU/ml) was also obtained from Sigma Chemicals, St. Louis, Mo. Purified CBHI (18.5 mg/ml) from Spezyme CP cellulase was prepared by Protein Labs (San Diego, Calif.). Enzyme protein contents were determined by the standard BCA method (Smith et al., 1985), and the activity of Novozyme188 was based on published data (Dien et al., 2008).

Enzymatic hydrolysis was performed according to NREL Laboratory Analytical Procedure LAP 09 in at least duplicates at 1% (w/w) glucan concentrations in 0.05M citrate buffer (pH=˜4.8) containing antibiotics (4000100 ml of 10 mg/ml tetracycline in 70% ethanol and 3000100 ml of 10 mg/ml cyclohexamide in DI water) in 125 ml Erlenmeyer flasks operated at 48±3° C. using a thermostated water bath shaker at ˜200 rpm (NREL, 1996). Substrate blanks without enzyme and enzyme blanks without substrate were run in parallel. Unless otherwise stated, digestibility was determined for Spezyme CP cellulase loadings of 6.5, 16.1, 32.2, and 129.0 mg of protein/g glucan in the raw biomass (corresponding to about 3.0, 7.5, 15, and 60 FPU/g of original glucan) supplemented with β-glucosidase at a CBU to FPU activity ratio of 2.0. Sugar release from commercial grade cellulose and birch wood and beech wood xylan were determined at the same protein loadings for comparison.

In addition, xylanase was also used with a lower CTB loading of 14.5 mg protein/g original glucan (about 7.5 FPU/g original glucan), with the same xylanase to cellulase protein ratios used as for the higher cellulase loading plus an additional ratio of 11. These xylanase loadings were designated as MX0.2, MX0.5, MX1, MX2, MX5, and MX11 for xylanase to cellulase protein mass ratios of 0.2, 0.5, 1, 2, 5, and 11, respectively, unless otherwise noted. The total amounts of protein for each cellulase and xylanase loading are summarized in Table 2.

TABLE 2
Total combined amounts of cellulase, β-glucosidase,
and xylanase protein per g glucan in unpretreated corn stover
for various xylanase to cellulase protein ratio.

| | Total amount of protein, | |
|---|---|---|
| | mg/g original glucan | |
| Xylanase to cellulase | 18.5 mg cellulase | 29.0 mg cellulase |
| protein mass ratio [designation] | (~7.5 FPU) | (~15 FPU) |
| 0.0:1 [MX₀] | 14.5 | 29.0 |
| 0.2:1 [MX_{0.2}] | 16.8 | 33.7 |
| 0.5:1 [MX_{0.5}] | 21.1 | 82.1 |
| 1:1 [MX₁] | 28.1 | 58.1 |
| 2:1 [MX₂] | 42.1 | 84.2 |
| 5:1 [MX₅] | 84.2 | 168.4 |
| 11:1 [MX₁₁] | 168.4 | NA |

For hydrolysis of corn stover with purified CBHI, the solids containing 1% (w/v) glucan were hydrolyzed for two hours at 50° C. for a CBHI loading of 15 mg/g glucan. The samples were analyzed for cellobiose and glucose. The equivalent glucose yield was defined as the ratio of total glucan equivalent hydrolyzed (0.9*(glucose+1.053*cellobiose)) to total potential glucan available in the pretreated solids. Similarly, the xylose yield was the ratio of total xylan hydrolyzed (0.89*xylose) to total potential xylan available in the pretreated solids.

To determine the amount of sugars generated during hydrolysis, liquid samples of about 700 μl were drawn at 24, 48, and 72 hours and then immediately filtered through 0.2 μm nylon filter vials (Alltech Associates Inc., Deerfield, Ill.), pipetted into 500 μl polyethylene HPLC vials (Alltech Associates Inc., Deerfield, Ill.), and kept refrigerated at 4° C. or frozen at −20° C. for longer times until analysis. Hydrolysis samples along with calibration standards were run on a Waters Alliance HPLC system (Model 2695, Waters Corporation, Milford, Mass.) employing Aminex HPX-87H and HPX-87P columns (Bio-Rad Laboratories, Hercules, Calif.).

The acetyl content of corn stover solids was determined per the NREL LAP 002 method using glacial acetic acid as a calibration standard (NREL, 2004).

After seventy-two hours of enzymatic hydrolysis, broths were centrifuged to separate undigested solids and insoluble lignin from the liquid. Then, the solid free liquid was incubated for one hour with four percent sulfuric acid at 121° C. in an autoclave along with sugar recovery standards. Thereafter, liquid was neutralized with CaCO₃, and the total amount of glucose and xylose analyzed by HPLC.

The percent yield of cellooligomers with a degree of polymerization>cellobiose, G3+, was calculated as: G3+=100*(glucose after post hydrolysis−glucose before post hydrolysis−1.053*cellobiose before post hydrolysis)/total potential glucose in pretreated solids. X2+, the percent yield of xylooligomers containing two or more xylose units, was calculated as: X2+=100*(xylose after post hydrolysis−xylose before post hydrolysis)/total potential xylose in pretreated solids. In which the yields after post hydrolysis were corrected for sugar degradation using a sugar recovery standard known in the art.

The effect of β-glucosidase supplementation on the digestibility of lignocellulosic biomass following pretreatment was investigated. One aspect of the study was directed to the addition β-glucosidase to commercial grade cellulose, amorphous cellulose, and corn stover solids pretreated with dilute sulfuric acid in a Parr reactor. Another aspect of the study was directed to the addition β-glucosidase after pretreatment of corn stover solids with SO₂. A mass loading of 16.1 mg of cellulase plus β-glucosidase protein/g original glucan was employed for Avicel cellulose and dilute sulfuric acid and SO₂ pretreated corn stover solids, and ten mg/g glucan (about five FPU) of cellulase plus β-glucosidase was added to amorphous cellulose. For all, β-glucosidase was added at activity ratios (CBU to FPU) of zero, one, two five, ten, and fifteen, unless otherwise stated, of beta-glucosidase to cellulase activity in addition to the CBU to FPU ratio of about 2.2 for Spezyme CP itself. The concentration of oligomers in the liquid after four hours of hydrolysis for amorphous cellulose and twenty-four hours of hydrolysis for Avicel and corn stover solids was determined as described above.

Hydrolysis of birchwood xylan was conducted at two different Multifect xylanase loadings of 7.0 and 14.0 mg/g xylan augmented with up to 10.0 mg of β-xylosidase protein per mg of xylanase protein. Furthermore, hydrolysis of AFEX pretreated corn stover was conducted at a mass loading of cellulase plus β-glucosidase of 16.1 mg/g original glucan augmented with up to 10.0 mg of β-xylosidase protein per mg of cellulase protein. The impact of β-xylosidase supplementation on sugar yields for SO₂ pretreated corn stover was only determined with a β-xylosidase to cellulase protein ratio of about 0.25. The release of sugar monomers and oligomers into the liquid was measured after seventy-two hours of hydrolysis, as described herein.

Hydrolysis of one percent (w/w) commercial grade cellulose with an equal mass of birch wood xylan [approximately eighty-six percent xylan] was performed in triplicate at a cellulase plus β-glucosidase loading of 16.1 mg/glucan. Hydrolysis was also performed at a similar cellulase loading but supplemented with β-xylosidase at a loading of 14 mg/g glucan. To evaluate the effect of xylose alone on cellulose hydrolysis, cellulose containing an equivalent amount of xylose (1.053*amount of xylan) was enzymatically hydrolyzed in parallel. Controlled experiments for cellulose hydrolysis without xylan, cellulose with β-xylosidase only, and a substrate blank containing cellulose plus xylan were run in parallel. Samples were collected four, twenty-four, forty-eight, and seventy-two hours after initiation of hydrolysis and analyzed for sugars. Liquids produced by seventy-two hours of hydrolysis of samples containing xylan were post hydrolyzed to determine the amount of xylooligomers as described herein.

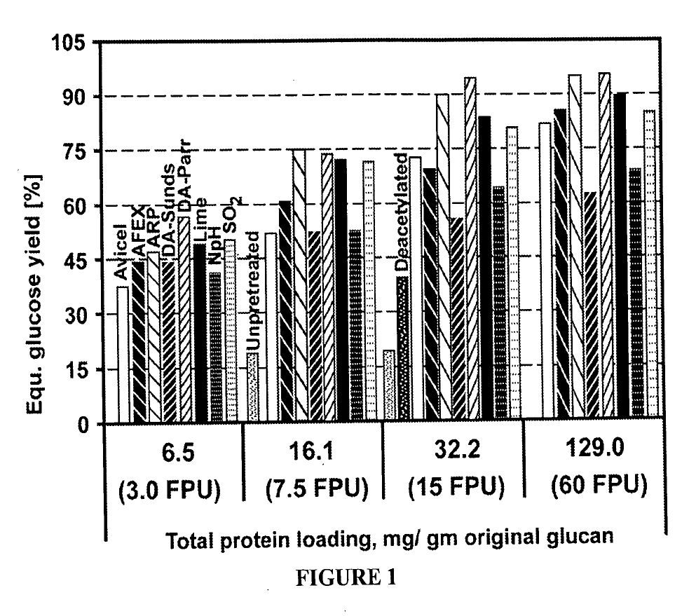

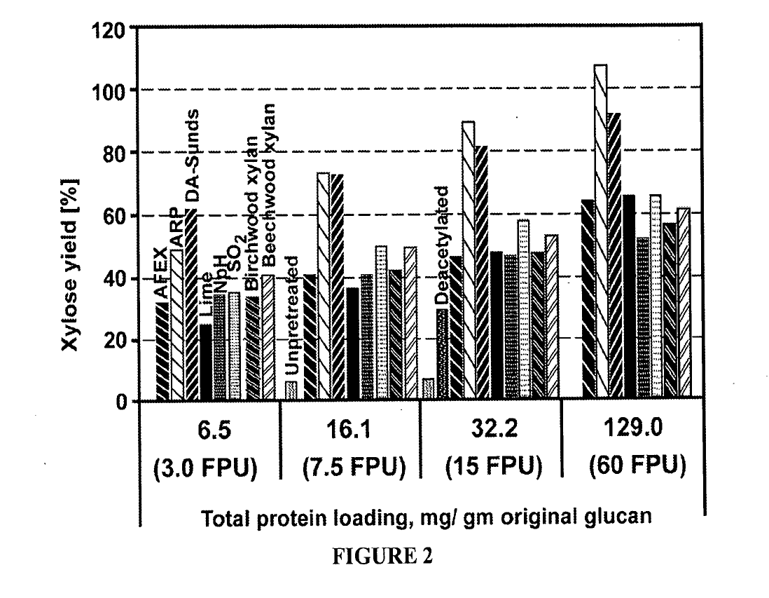

FIG. 1 is a graphical representation of equivalent glucose yields compared to mass protein loadings after seventy-two hours of digestion for Avicel PH 101, unpretreated and deacetylated corn stover, and corn stover solids following pretreatment by leading technologies. Equivalent glucose yield is defined as grams of glucose (plus 1.053*cellobiose) per grams of glucan in the solids before hydrolysis. FIG. 2 is a graphical representation of Xylose yields compared to mass protein loadings after seventy-two hours of digestion for pure xylan, unpretreated and deacetylated corn stover, and corn stover solids prepared by leading pretreatment technologies

Accordingly, and as shown in FIGS. 1 and 2, glucose release was very low for unpretreated corn stover (<20%) at a protein loading of cellulase plus β-glucosidase of 32.2 mg of/g original glucan, and generally, all pretreatments resulted in higher glucose removal than possible from commercial grade cellulose at lower enzyme loadings. However, at higher protein loadings, sugar release was somewhat lower for solids prepared by dilute sulfuric acid in the Sunds system and by neutral pH pretreatment than from commercial grade cellulose. On the other hand, solids resulting from dilute sulfuric acid pretreatment in the Parr reactor (*severity [log R0]=3.06) showed much higher digestibility than for solids pretreated with dilute sulfuric acid in the Sunds system (severity [log R0]=2.53) at all protein loadings. The fractional release of xylose (FIG. 2) was much lower from pretreated solids than for glucose (FIG. 1) for all pretreatments. Yet, xylose yields at a high protein loading of 129 mg/g original glucan ranged from 60% to nearly complete saccharification.

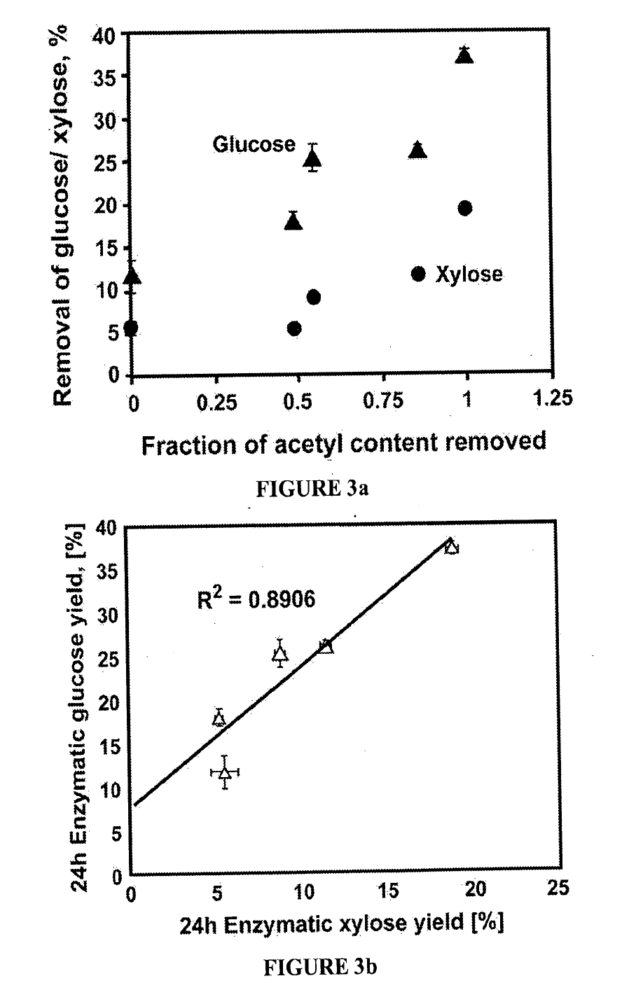

FIG. 3(a) is a graphical representation demonstrating the effect of deacetylation on glucan and xylan removal after twenty-four hours of enzymatic hydrolysis for an enzyme loading of 32.2 mg. FIG. 3(b) is a graphical representation comparing twenty-four hour enzymatic glucose yield compared to xylose yield for untreated and deacetylated corn stover. Although release of sugars was lower with removal of acetyl groups (FIGS. 1 and 2) than for any of the pretreatments, there was a nearly linear relationship between glucose yields and acetyl removal for a protein mass loading of 32.2 mg/g glucan (FIG. 3a). Furthermore, glucose yields approximately doubled over the range of modest acetyl removal achieved, and xylose removal was significantly enhanced as well. In addition, a linear relation was observed between xylose and glucose removal for untreated and deacetylated corn stover (FIG. 3b).

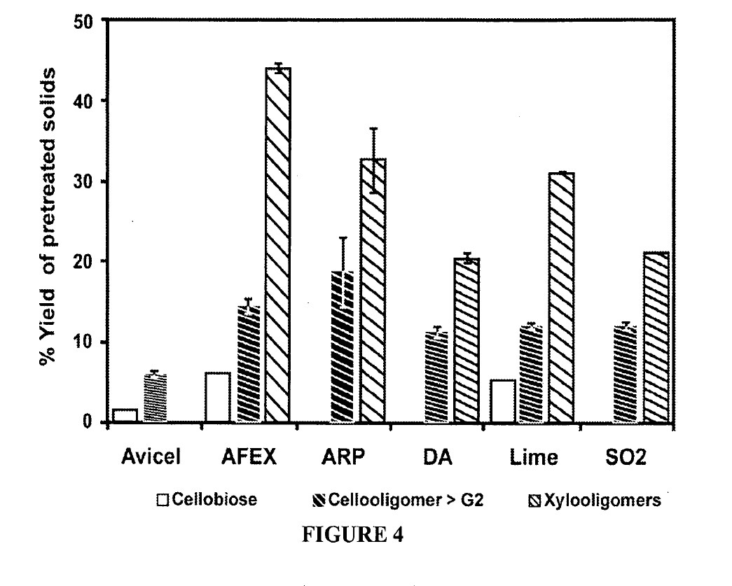

FIG. 4 is a graphical representation showing yields of cellobiose, higher cellooligomers, and xylooligomers as determined by post hydrolysis following enzymatic hydrolysis for seventy-two hours at a cellulase plus β-glucosidase protein loading of 16.1 mg/g original glucan in accordance with the disclosure. Using only Spezyme CP cellulase that is low in xylanase activity is expected to result in the incomplete removal of xylan, as confirmed by FIGS. 1 and 2. The low xylanase activity of cellulase could also explain why xylooligomer yields as determined by post hydrolysis were as high as forty-five percent following seventy-two hours of hydrolysis, (FIG. 4) for a protein loading of 16.1 mg/g original glucan.

Surprisingly, at lower cellulase loadings substantial amounts of cellobiose and particularly higher degree of polymerization cellodextrins (>cellobiose) were also found in the hydrolyzates, with yields of up to twenty percent after seventy-two hours of hydrolysis, depending upon the substrate and pretreatment. The cause of this substantial cello-oligomers accumulation could be adsorption/inactivation of cellulase components and/or β-glucosidase on lignin. Although several studies have reported the beneficial effect of β-glucosidase supplementation on glucan digestibility, no universally effective loading of (β-glucosidase has been defined, perhaps due to variations in β-glucosidase activity in cellulase and the effects of the substrates used. Therefore, more detailed investigations appear warranted on whether the effectiveness of β-glucosidase supplementation is influenced by pretreatment choice and the type of substrate as well as enzymatic hydrolysis conditions such as the duration of hydrolysis, temperature, and substrate loading.

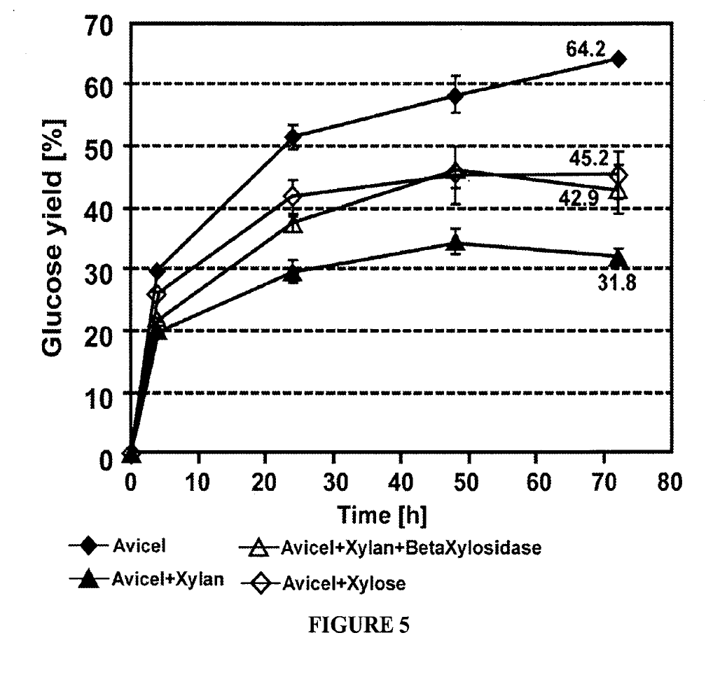

Referring now to FIG. 5, an aspect of the disclosure is the addition of β-xylosidase to effect hydrolysis of cellulose. Although several studies reported inhibition of cellulase activity by monomeric sugars and cellobiose, limited information is available on the effect of higher cellooligomers and xylooligomers. For example, it has been reported that effluent exiting from low liquid ammonia recycled percolation (LLARP) pretreatment strongly inhibited cellulase and microbial activities, but the report was unclear whether high amounts of xylooligomers or solubilized lignin in the liquid were responsible. Therefore, Spezyme CP supplemented with β-glucosidase at a 1:2 ratio, respectively, was added to equal concentrations of 10 g/L for both xylan and commercial grade cellulose, wherein the seventy-two hour glucose yield was about half that of the control.

As shown in FIG. 5, adding xylan (10 g/l), xylose (12 g/l), and β-xylosidase affects the hydrolysis of Avicel glucan with at a cellulase plus β-glucosidase protein loading of 16.1 mg/g glucan. The concentration of xylose in the hydrolyzate was measured to be 5.5 g/l. However, supplementing cellulase with β-xylosidase at a protein mass loading of 14 mg/g glucan improved yields by about thirty-four percent, resulting in about the same performance as realized when an amount of xylose equivalent to the xylan concentration originally employed, 12.5 mg/ml, was added initially. Yet, only about 6.7 mg/ml of xylose was released by hydrolysis of the mixture with β-xylosidase supplementation at a protein loading of 14 mg/g glucan. Furthermore, in the initial period of cellulose hydrolysis in the presence of xylan, cellobiose appeared in higher amounts than for the control, suggesting inhibition of β-glucosidase activity by xylooligomers and/or xylan. However, cellobiose virtually disappeared after twenty-four hours of hydrolysis, and β-xylosidase had very low cellulase activity (data not shown). Thus, these observations confirm an aspect of the disclosure that xylooligomers inhibit cellulase action, and glucose and xylose release may be significantly enhanced by β-xylosidase supplementation. As may be apparent to those having ordinary skill in the art, the benefits of adding β-xylosidase are expected to vary with the amount of xylan left in the solids, which is in turn affected by the type of pretreatment and substrate. In addition, the choice of enzyme and enzyme loadings are also expected to be important to glucose and xylose release.

Cellobiose produced by the action of cellobiohydrolases on cellulose is well recognized by those having ordinary skill in the art to strongly inhibit cellulase action, and several studies reported the effects of adding β-glucosidase on cellulose hydrolysis. The impact, however, of β-glucosidase supplementation on cellodextrin release after enzymatic hydrolysis and inhibition by cellodextrins with degrees of polymerization greater than for cellobiose have seldom been studied. As shown in FIG. 4, despite adding amounts of β-glucosidase consistent with literature recommendations, yields of higher DP cellooligomers were high for pure cellulose and lignin containing substrates, even though the yield of cellobiose was lower. This is consistent with reports that the effectiveness of (β-glucosidase drops with increasing chain lengths of soluble cellooligomers. Furthermore, β-glucosidase is well known to lose activity by exposure to heat and agitation, nonproductive binding with lignin, and the effect of lignin degradation compounds. In accordance with the methods of the disclosure, the effectiveness of a given β-glucosidase loading may vary with the type of substrate, pretreatment method, substrate loading, and time of hydrolysis.

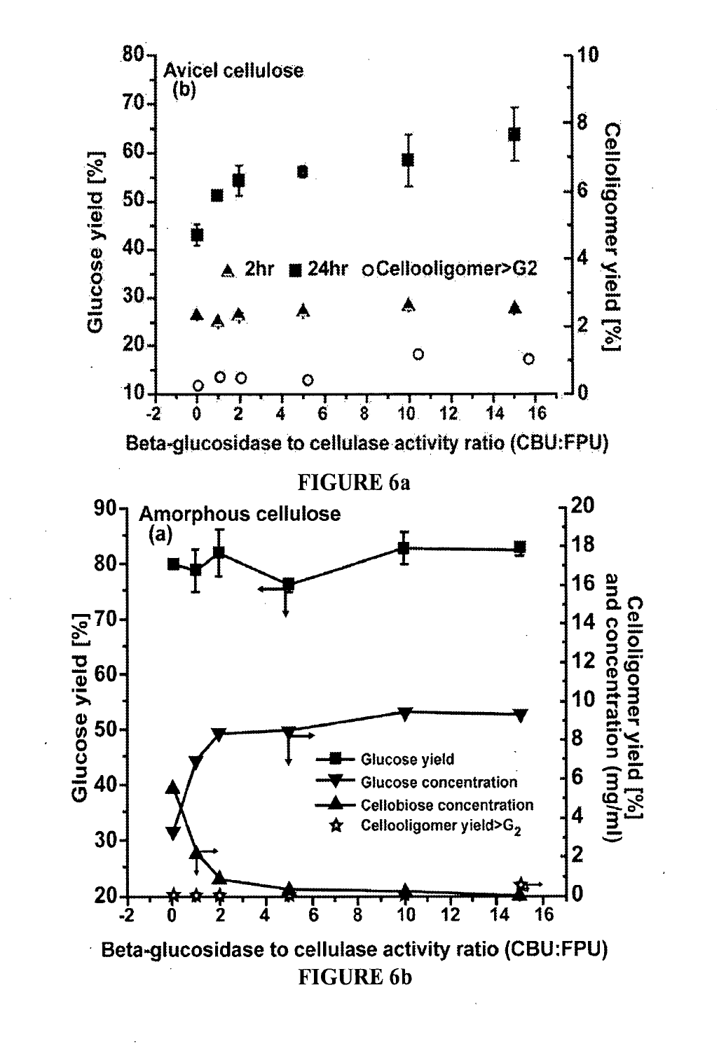

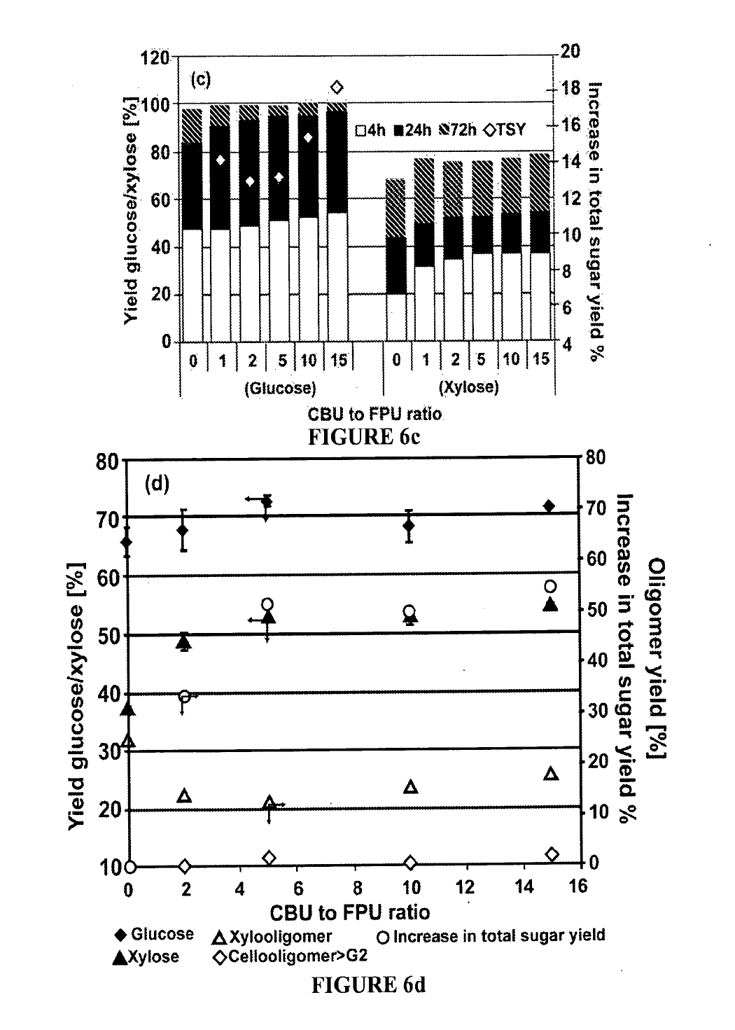

FIG. 6 shows the effect of jβ-glucosidase supplementation on sugar release and oligomer yields at a fixed cellulase plus β-glucosidase mass loading of 10.0 mg/g glucan for (a) amorphous cellulose after four hours of hydrolysis and with a mass loading of 16.1 mg/g glucan of cellulase plus β-glucosidase for (b) Avicel PH 101 after two and twenty-four hours (c) dilute acid pretreated corn stover after seventy-two hours, and (d) SO₂ pretreated corn stover after twenty-four hours in a Parr reactor. Contradictory to expectations, increasing β-glucosidase activity did not significantly enhance cellulose hydrolysis to glucose and cellobiose for amorphous cellulose, even though it did increase cellobiose conversion to glucose up to a CBU to FPU activity ratio of two, as shown in FIG. 6a. Furthermore, β-glucosidase supplementation beyond that ratio reduced cellobiose concentrations to virtually zero, but the yield of higher cellooligomers remained at about 0.5 percent, even at a very high ratio of β-glucosidase to cellulase activity. Although β-glucosidase supplementation did not have much effect on the initial rate of hydrolysis for Avicel PH101, as shown in FIG. 6b, it had a major impact on the twenty-four hour glucose yield, and surprisingly, the yield of higher cellooligomers (>G2) was around one percent to two percent even with very high β-glucosidase supplementations (CBU to FPU ratio of approximately fifteen). β-glucosidase supplementation had a small effect on the initial rate of glucose release and a major effect on initial xylose release for dilute sulfuric acid pretreated corn stover, as shown in FIG. 6c, but had a significant impact on twenty-four hour yields of both glucose and xylose (increase in total sugar yield of about thirty-nine percent).

As further shown in FIG. 6, β-glucosidase supplementation had a noticeable effect on total sugar yields in the first twenty-four hours but only increased glucose and xylose yields by 2.8 percent and fifteen percent, respectively, after seventy-two hours. Therefore, for dilute acid pretreated corn stover prepared with the Parr reactor, a CBU to FPU activity ratio of one appears adequate for achieving nearly theoretical glucose yields even at a low cellulase loading of 16.1 mg/g glucan. Supplemental β-glucosidase had little effect on glucose release at a cellulase loading of 32.2 mg/g glucan (fifteen FPU) with a substrate loading of one percent (w/v) glucan (data not shown). For SO₂ pretreated corn stover, twenty-four hour glucose yields increased with increasing β-glucosidase supplementation up to a ratio of five (by ten percent) and then remained constant, as shown in FIG. 6d, and the overall increase in total glucose plus xylose yields was about 50% at a CBU to FPU activity ratio of five. It is interesting to note that β-glucosidase supplementation resulted in a thirty percent decrease in yield of xylooligomers to about twenty-five percent of the total xylan available in the pretreated solids, and the yield of cellooligomers was around two percent, even with a very high β-glucosidase supplementation. Therefore, β-glucosidase does not hydrolyze all of the higher cellodextrins, and cellulose crystallinity, type of pretreatment, and hydrolysis duration may influence the effectiveness of β-glucosidase supplementation, as suggested in the literature. The increase in xylose yields with β-glucosidase supplementation may result from xylanase activity in Novozyme188.

Enzymatic hydrolysis requires synergy between endo-xylanase and β-xylosidase for unsubstituted xylan and among xylanase components and accessory enzymes for substituted xylan. However, although the role of β-xylosidase has some similarities to that for β-glucosidase, it is more effective in hydrolyzing higher DP soluble xylans than β-glucosidase is in hydrolyzing higher DP cellooligomers. As shown previously in FIG. 4, the yield of xylooligomers after enzymatic hydrolysis with just cellulase and β-glucosidase can be as high as forty-five percent, as observed for solids containing one percent (w/w) glucan, and depends on type of pretreatment, possibly due to limited β-xylosidase activity in Spezyme CP.

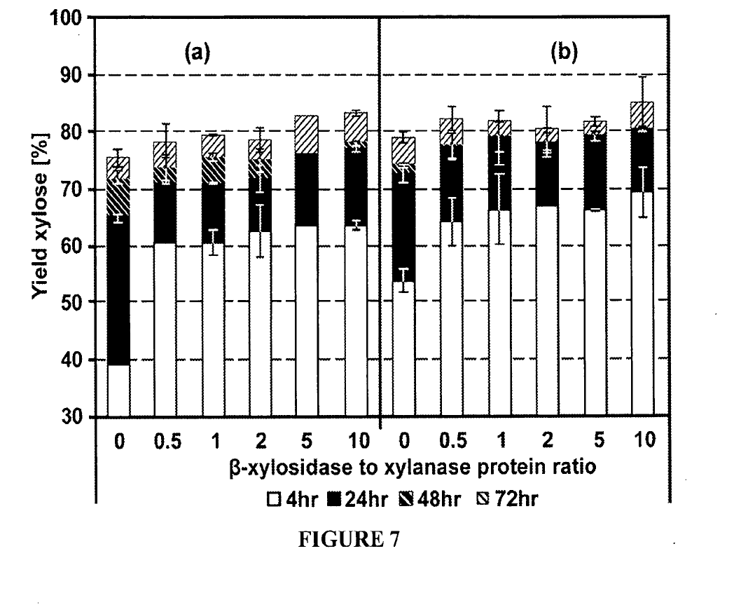

In accordance with the method of the disclosure, birch wood xylan was hydrolyzed at two xylanase mass protein loadings of seven mg and fourteen mg/g xylan with β-xylosidase supplementation up to ten mg/mg of xylanase protein. FIG. 7 summarizes xylose monomer yields for samples collected after four, twenty-four, forty-eight, and seventy-two hours. This data showed that β-xylosidase supplementation did not increase longer time xylose yields significantly beyond a protein mass ratio of one, possibly due to the high activity of β-xylosidase in Multifect xylanase. In accordance with the disclosure, supplementation with β-xylosidase, had a substantial effect on the initial rate of xylose release for each xylanase mass loading, with about a fifty-five percent and twenty percent increase for seven mg and fourteen mg of xylanase protein, respectively, in the first four hours. Furthermore, β-xylosidase supplementation dropped xylooligomers yields to essentially zero percent (data not shown) from five percent with a xylanase mass loading of seven and to zero percent from 1.7% with a xylanase mass loading of fourteen mg/g xylan. Although higher xylanase loadings and β-xylosidase supplementation both increased initial xylose yields significantly, seventy-two hour yields were quite similar at both xylanase mass loadings with β-xylosidase supplementation.

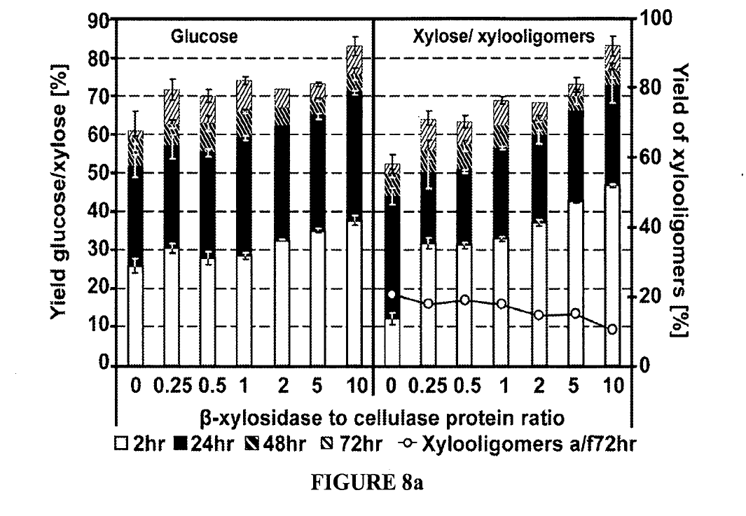

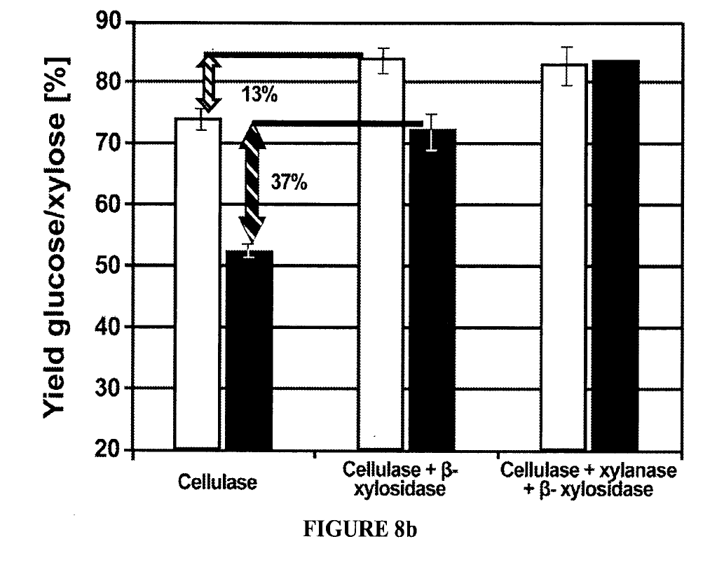

With reference to FIG. 8, β-xylosidase supplementation impacts glucan and xylan hydrolysis for substrates containing appreciable amounts of xylan and lignin. In accordance with the methods of the disclosure, AFEX pretreated corn stover was hydrolyzed at a cellulase plus β-glucosidase mass loading of 16.1 mg/g original glucan with up to ten mg of β-xylosidase supplementation per mg of cellulase in accordance with the disclosure. Similarly, SO₂ pretreated corn stover was hydrolyzed at a cellulase plus β-glucosidase loading of 16.1 mg/g with 0.25 mg of β-xylosidase/mg of cellulase and with an equal amount of xylanase protein as cellulase plus β-xylosidase. As shown in FIG. 8a, β-xylosidase supplementation increased four hour glucose and xylose releases by up to forty-six percent and two-hundred eighty-five percent, respectively, at high supplementation ratios. These results for glucan hydrolysis support earlier evidence that xylooligomers significantly inhibit cellulase activity. In addition, seventy-two hour glucose and xylose yields at a protein mass ratio of β-xylosidase to cellulase of 0.25 were enhanced by about eighteen percent and twenty-three percent, respectively, although β-xylosidase supplementation enhanced sugar release, a significant yield of xylooligomers (about ten percent) was still found after seventy-two hours of hydrolysis even for very high β-xylosidase supplementation, as shown in FIG. 8a.

Referring to FIG. 8b, for SO₂ pretreated corn stover with β-xylosidase supplementation, glucose and xylose release increased by about thirteen percent and thirty-seven percent, respectively. Although supplementation of the enzyme mixture containing cellulase, β-glucosidase, and xylanase with β-xylosidase enhanced xylose release by sixteen percent, a negligible increase in glucose release was observed, suggesting that β-xylosidase supplementation may not be beneficial with a high xylanase loading. Thus, β-xylosidase alone does not appear to be sufficient to hydrolyze high DP soluble xylooligomers, and supplementation with both xylanase and β-xylosidase appears desirable to realize high monomeric xylose yields, as found for pure xylan and AFEX and SO₂ pretreated corn stover.

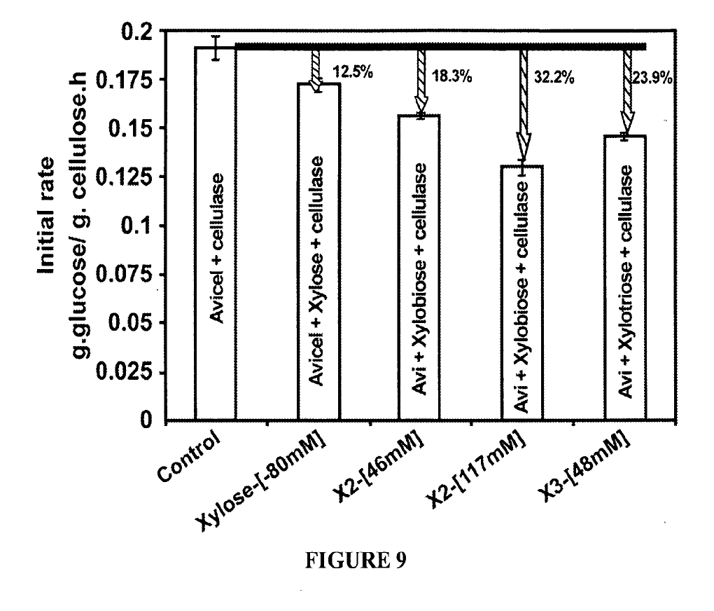

As demonstrated by the examples herein, significant amounts of xylooligomers were measured in the hydrolyzates after enzymatic hydrolysis. Furthermore, these xylooligomers have been found in the liquor from pretreatment prior to enzymatic hydrolysis, with the amounts depending upon the type of pretreatment, substrate, and substrate loading. Furthermore, such xylooligomers can slow glucose and xylose release. Referring now to FIG. 9, quantification regarding how oligomers concentration and chain length affect this inhibition was demonstrated. In accordance with an aspect of the disclosure, 12.5 mg/ml (eighty mM xylose equivalent) of xylose, 6.6 mg/ml (forty-six mM xylose equivalent) and 16.6 mg/ml (one-hundred seventeen mM xylose equivalent) of xylobiose, and 6.6 mg/ml (forty-eight mM xylose equivalent) of xylotriose were added to two ml centrifuge tubes containing thirty mg of commercial grade cellulose in 1.5 ml of liquid at a cellulase loading of thirty FPU/g glucan, and initial hydrolysis rates were measured. Xylose alone at this concentration inhibited yields by about ten percent, but about half as much xylobiose, forty-six mM of xylose equivalent, reduced glucose release by eighteen percent. Furthermore, glucose release dropped by about thirty-two percent at a higher xylobiose loading of one-hundred seventeen mM of xylose equivalent. Adding almost half of the xylose concentration and about the same as the lower concentration of xylobiose, xylotriose (forty-eight mM xylose equivalent) reduced glucose release even more, by about twenty-four percent. These results show that enzyme inhibition increases with chain length and concentration for these three species.

In accordance with the methods of the disclosure, glucose and xylose release data were developed for solids prepared by leading pretreatment options that span a range of pH values to better understand what factors drive the need for high enzyme doses to realize high yields. It was observed that about seventy percent to eighty percent of the glucan was digestible at a cellulase plus β-glucosidase mass loading of 16.1 mg/g glucan and that glucose release was better from all pretreatments than from commercial grade cellulose at these lower enzyme loadings. Xylose yields, however, were much lower and remained lower even with very high cellulase mass loadings. Analysis of the hydrolyzate following pretreatment and in the liquid after seventy-two hours of enzymatic hydrolysis showed an appreciable amount of oligomers, with the former containing large amounts of xylooligomers. Furthermore, enzymatic hydrolysis was slower when xylooligomers were present, suggesting that they strongly inhibited cellulase activity and possibly endo-xylanase activity as well. Supplementation of cellulase with β-xylosidase and xylanase improved sugar release significantly, with their effectiveness depending on the type of substrate and pretreatment and the length of time for hydrolysis. In addition, xylose, xylobiose, and xylotriose were shown to inhibit enzymatic hydrolysis, and the degree of inhibition was found to increase with chain length and concentration for these three compounds.

In yet further experiments and analyses the disclosure demonstrates the advantages and utility of the invention. Pretreated corn stover solids were enzymatically hydrolyzed at fixed CTB mass loadings of 14.5 and 29.0 mg/g glucan in unpretreated corn stover with xylanase supplementation to various degrees for a total of 72 h to establish trends in enzyme effectiveness. However, longer hydrolysis times (e.g., 7 days) would likely be employed commercially to capitalize on the additional sugar release expected.

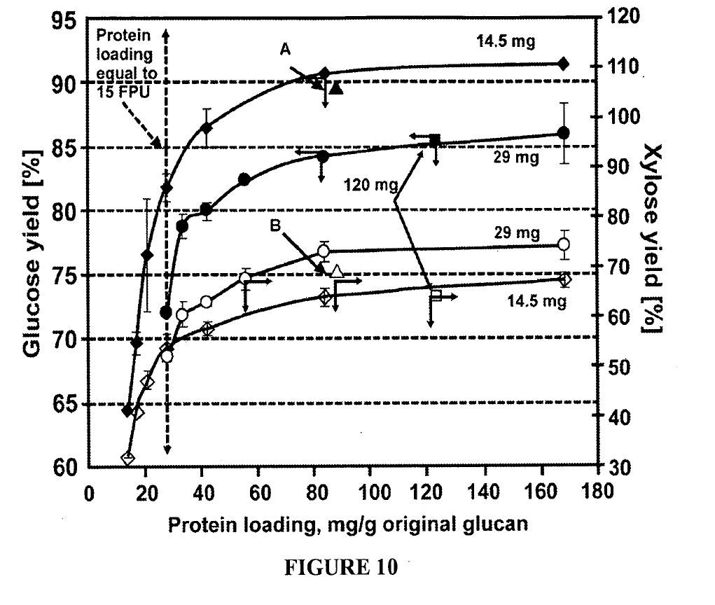

As AFEX pretreatment retains virtually all the carbohydrates intact in biomass (Alizadeh et al., 2005; Murnen et al., 2007; Teymouri et al., 2005), higher sugar yields can be realized with lower protein demands by reconstituting the enzyme cocktail to include other enzymes than just cellulase (Hespell et al., 1997). As shown in FIG. 10, xylanase supplementation of just ⅕th (MX0.2) of the cellulase mass loading increased 4 glucose yields by about 9% and 8.5% at 14.5 and 29.0 mg of cellulase plus b-glucosidase mass loading, respectively, and xylose yields by 30% and 15% at the corresponding loadings. Furthermore, 72 h glucose and xylose release continued to increase with further xylanase supplementation. The increase in glucose and xylose yields (40.5% and 101%, respectively) at a xylanase supplementation mass ratio of 5 (MX5) was much higher at the lower cellulase plus b-glucosidase mass loading of 14.5 mg than the increase of 19.4% and 49.1%, respectively, at 29.0 mg cellulase.

Furthermore, glucan digestibility at a cellulase plus b-glucosidase mass loading of 14.5 mg and a xylanase supplementation mass ratio of 1 (MX1) for a total of 29 mg of protein was ˜10% higher than digestibility with cellulase alone with the same total mass of protein (15 FPU) and almost equal to the digestibility for a cellulase plus b-glucosidase mass loading of 120 mg (60 FPU). Thus, adding more xylanase was more effective than adding more cellulase.

Xylose release at a cellulase plus b-glucosidase loading of 14.5 mg and xylanase supplementation mass ratio of 1 was almost equal and a bit lower than at the cellulase mass loading of 29 and 120 mg, respectively. Yet, it was also observed that >90% glucan digestion was obtained at a cellulase plus b-glucosidase loading of just 14.5 mg for a xylanase supplementation mass ratio of 5 (MX5; total protein ┐ 85 mg), but xylan digestibility was still <80% for both cellulase mass loadings for a xylanase supplementation mass ratio of 5 (total protein 84.0 and 168 mg, respectively).

Thus, because soluble xylooligomers might inhibit enzyme action (Kumar and Wyman, 2009), 14 mg b-xylosidase was added per g of glucan for a cellulase plus b-glucosidase mass loading of 14.5 mg at a xylanase supplementation mass ratio of 5 (MX5) (total protein ˜88.0 mg/g glucan in unpretreated biomass). In this case, xylose release was enhanced by about 9%, even though glucose release was virtually unaffected. Thus, even though xylose yield increased to ˜90% for supplementation of cellulase with both xylanase and b-xylosidase, small amounts of other xylan debranching enzymes (such as feruloyl esterase) can be beneficial to attain complete hydrolysis of xylan in AFEX pretreated corn stover because xylan branching inhibits xylanase and probably cellulase activity as well (Anand and Vithayathil, 1996; Kormelink and Voragen, 1992; Kumar and Wyman, 2009b; Suh and Choi, 1996).

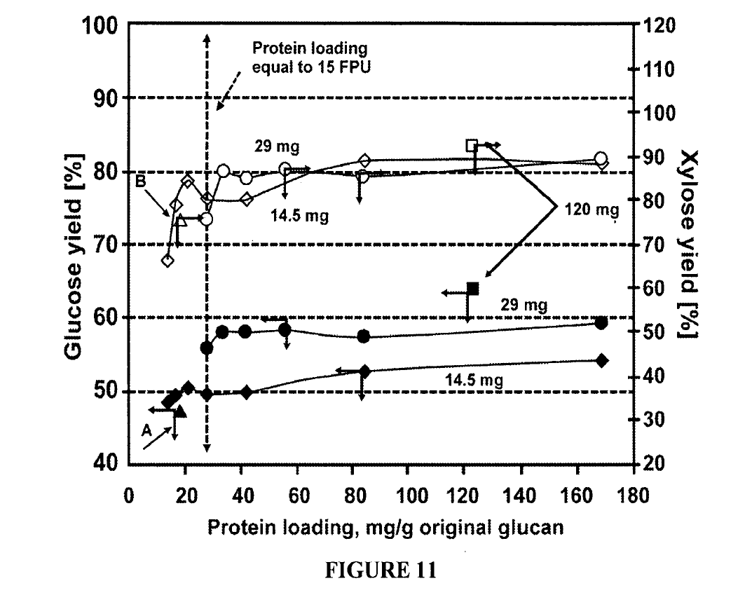

Although ARP pretreatment typically removes about 50% of the xylan and 80% of the lignin from corn stover (Kim and Lee, 2005; Kim et al., 2006; Wu and Lee, 1997), the xylan content in pretreated solids was still about 18% due to high lignin removal. As shown in FIG. 11, xylanase supplementation enhanced digestion less for ARP pretreated corn stover than for AFEX. In fact, a xylanase supplementation mass ratio of 5 (MX5) increased glucose yields by about 30.0% and 9.0% at cellulase mass loadings of 14.5 and 29.0 mg, respectively, and xylose yields by about 60.5% and 31.7% in the same order of cellulase loadings. Furthermore, glucan and xylan digestion was much higher for the lower cellulase mass loading of 14.5 mg. However, glucan and xylan digestion for both cellulase loadings were still lower than for a cellulase plus b-glucosidase mass loading of 120 mg corresponding to 60 FPU/g original glucan (95.2% and 98% for glucan and xylan, respectively). Although experiments were not run, a low level of b-xylosidase supplementation could possibly improve xylose and glucose release further.

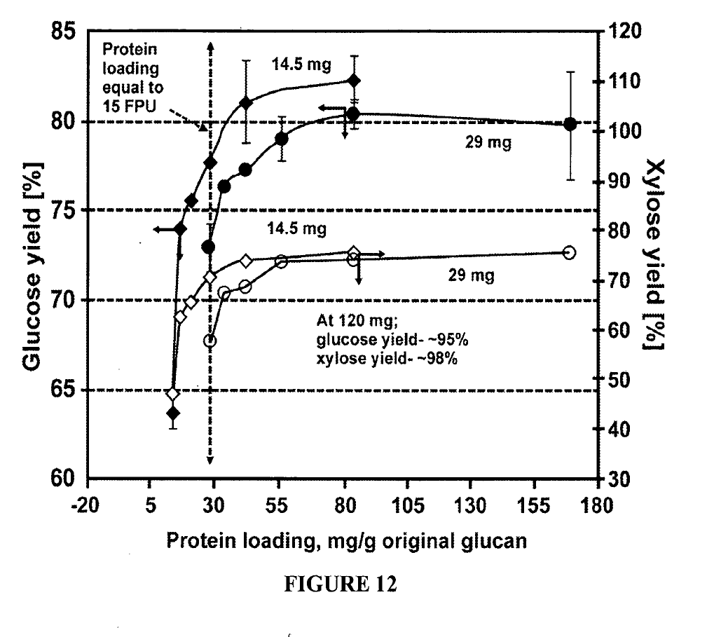

To evaluate how lower pretreatment severity impacted enzyme effectiveness, corn stover was pretreated with dilute acid at a low severity level of log R0=2.53 using the Sunds hydrolyzer (Metso Paper USA, Inc., Norcross, Ga., USA) at NREL for comparison to results for a high severity of about 3.06 reported elsewhere (Lloyd, 2005; Lloyd and Wyman, 2005). As is typical, severity is defined as R₀=t*exp ((T−100)/14.73) with t the time in minutes and T the temperature in ° C. (Lloyd and Wyman, 2005; Overend and Chornet, 1987). In this case, about 94% and 27% of the original glucan and xylan, respectively, were retained in the solids from the Sunds reactor. As shown in FIG. 12, digestion of these solids was much lower at a cellulase mass loading of 14.5 mg/g original glucan than reported in other studies with solids resulting from dilute acid pretreatment of corn stover with a Parr reactor (Lloyd and Wyman, 2005). Furthermore, although xylanase supplementation did not increase glucose release much (3-8%) for the two fixed cellulase mass loadings, xylose release increased by 33.6% and 12.9% for cellulase plus b-glucosidase mass loadings of 14.5 and 29.0 mg, respectively, for a xylanase supplementation mass ratio of 5 (MX5). At equal protein levels, sugar yields were higher for a mass loading of 120 mg of just cellulase plus beta-glucosidase/g glucan in unpretreated corn stover than possible with xylanase supplementation at cellulase loadings of 14.5 and 29.0 mg. bxylosidase supplementation increased xylose yield by about 13.6% and had a negligible effect on glucose release.

Based on these results and data reported elsewhere for dilute acid pretreatment (Lloyd and Wyman, 2005; Yang and Wyman, 2004), digestion is poor for less severe pretreatment, suggesting that disruption of the biomass complex network appears more important than removing/retaining xylan, lignin, or both. For example, the acetyl content in pretreated corn stover solids decreased with severity for dilute acid pretreatment from about 1.1% for the Sunds reactor (180° C., 1.5 min, 3.0% acid, log R₀=2.5) to 0.2% (160° C., 20 min, 0.5% acid, log R₀=3.1) and 0.3% (140° C., 40 min, 1.0% acid. log R₀=2.8). Presumably other internal linkages are disrupted to a greater extent at elevated severity. Gupta and Lee reported that the digestibility of solids from ammonia recycle pretreatment increased with pretreatment temperature, even though the compositions of pretreated solids prepared at different temperature were almost the same (Gupta et al., 2008).

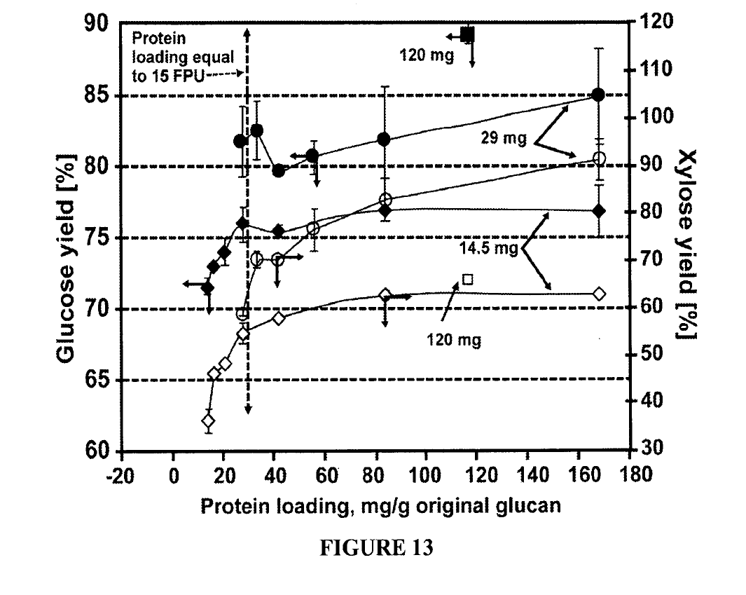

Similar to other alkaline methods, lime pretreatment leaves a major portion of the initial xylan in the solids (Chang et al., 1997; Kaar and Holtzapple, 2000), and consequently, enzymes must release xylose and other sugars in hemicellulose from the solids in addition to glucose to realize high total sugar yields. However, Kim and Holtzapple reported a xylan digestibility of only about 50% for lime pretreated corn stover solids at a cellulase loading of 15 FPU (Kim and Holtzapple, 2005), and higher mass loadings of cellulase with just b-glucosidase resulted in xylose yields below 75% (Kumar and Wyman, 2009b). The impact of supplementing cellulase with xylanase on xylose and glucose release is shown in FIG. 13 for lime pretreated corn stover at the same two fixed cellulase plus b-glucosidase mass loadings as employed previously.

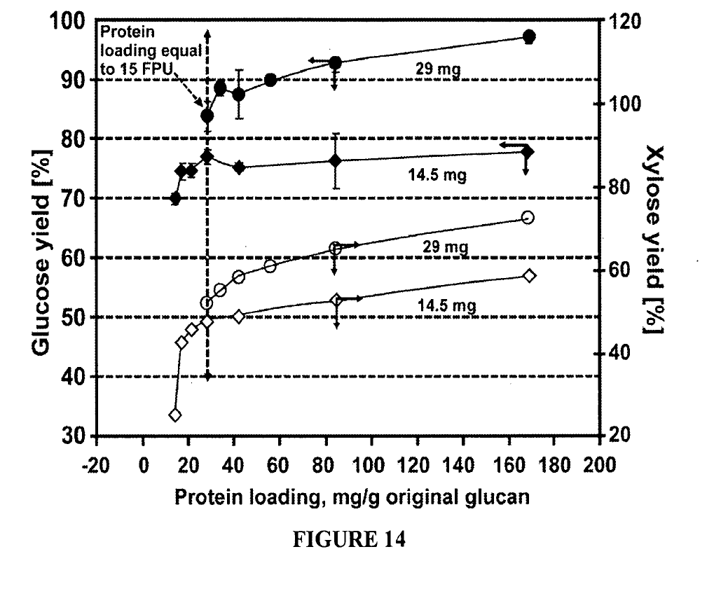

As shown in FIG. 14, xylanase supplementation at a mass ratio of 5 (MX5) resulted in modest 10% and 15% increases in glucose yields for cellulase plus b-glucosidase mass loadings of 14.5 and 29.0 mg, respectively, but much higher increases in xylose yields of 110% and 39%, respectively.

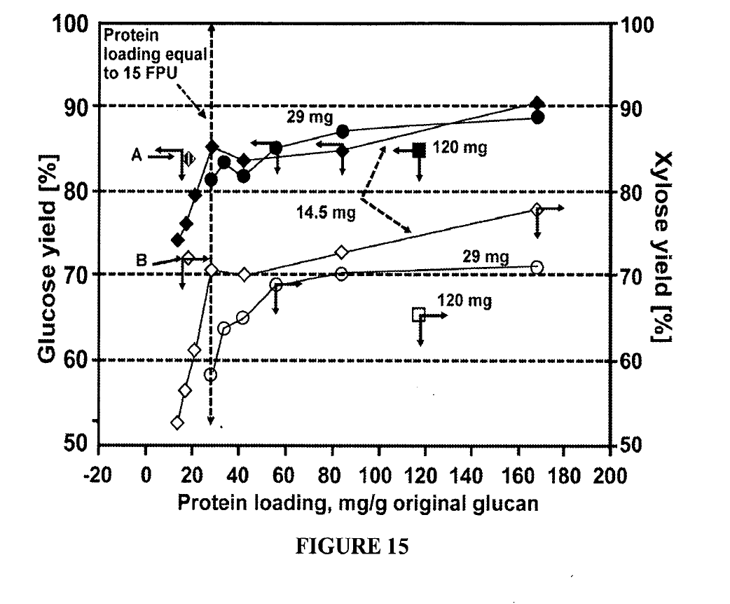

Xylanase supplementation significantly increased glucose and xylose release from SO₂ pretreated corn stover solids that contained about 11% xylan. As shown in FIG. 15, xylanase supplementation at a mass ratio of 5 (MX5) resulted in 15% and 9% increases in 72 h glucose yields for cellulase plus b-glucosidase mass loadings of 14.5 and 29.0 mg, respectively, and higher increases in xylose yields of 39% and 22%, respectively. However, for a cellulase mass plus b-glucosidase loading of 14.5 mg, glucose and xylose release continued to increase beyond the xylanase mass supplementation ratio of 5, as shown in FIG. 15. At an equal amount of total protein loading of 29.0 mg, as shown by the vertical dashed line, the 72 h release of glucose and xylose was higher when xylanase was added to a lower cellulase mass loading of 14.5 mg/g glucan in unpretreated corn stover than for a higher cellulase plus b-glucosidase loading without xylanase supplementation. At an equal total protein mass loading, glucose and particularly xylose release were higher for a cellulase mass loading of 14.5 and 29.0 mg with xylanase supplementation than for using 120 mg of just cellulase and b-glucosidase.

In additional experiments, using b-xylosidase at a loading of 14.0 mg/g glucan with cellulase at a mass loading of 14.5 mg increased glucose and xylose release by 12% and 35%, respectively, the same as gained by a xylanase mass supplementation ratio of 1. Furthermore, adding b-xylosidase to cellulase plus b-glucosidase at a mass loading of 14.5 mg supplemented with xylanase at a mass ratio of 1 increased glucose and xylose release by 15% and 58%, respectively. Thus, a significant amount of soluble xylooligomers apparently formed during enzymatic hydrolysis of SO2 pretreated corn stover that b-xylosidase effectively removed.

Synergism is often defined as the ratio of the rate or yield of a single product released by the simultaneous action of enzymes to the sum of rate or yield of these products when produced by the action of individual enzymes when used separately in the same amounts as in the mixture. However, because this study followed release of two products, glucose and xylose, over a range of loadings of xylanase, a new term xylanase leverage was defined as the ratio of the percent increase in glucose release to the percent increase in xylose release to illustrate how releasing additional xylan enhanced glucan hydrolysis. As summarized in Table 3, the degree of xylanase leverage on cellulase effectiveness varied with the type of pretreatment.

TABLE 3
The degree of leverage between cellulase and xylanase
for two fixed cellulase plus β-glucosidase mass loadings
with multiple xylanase supplementations.

| | % Residual | % | | |
|---|---|---|---|---|
| | xylan in | Acetyl | Degree of leverage* | |
| Pretreatment | pretreated solid | content | @ 7.5 FPU | @ 15 FPU |
| Untreated | 22.8 | 2.51 | — | — |
| AFEX | 22.8 | 1.69 | 0.390 | 0.473 |
| ARP | 17.9 | 0.30 | 0.474 | 0.311 |
| Dilute acid (Sunds) | 9.5 | 1.13 | 0.221 | 0.298 |
| Lime | 26.4 | 0.20 | 0.099 | 0.001 |
| Controlled pH | 16.2 | 1.14 | 0.089 | 0.680 |
| SO₂ | 11.6 | 1.15 | 0.427 | 0.349 |

*Leverage = % increase in glucan digestion/% increase in xylan conversion.

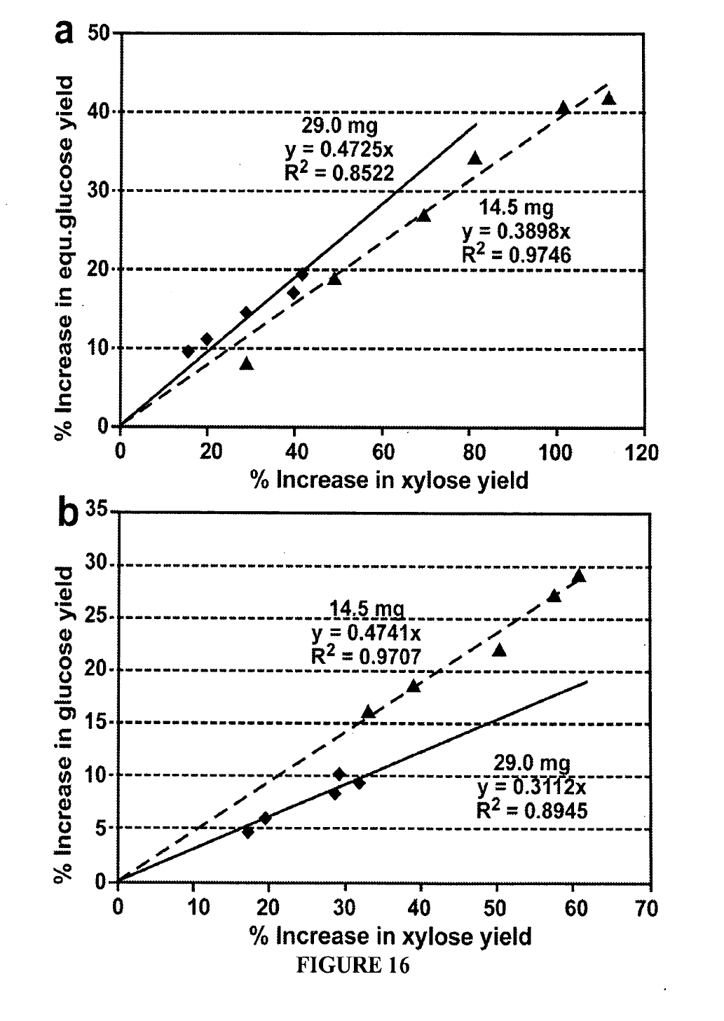

As shown in FIG. 16a and b for AFEX and ARP pretreated corn stover solids, respectively, a strong linear relation (R2=0.97 and 0.85 for AFEX and 0.97 and 0.89 for ARP corn stover solids for 14.5 and 29 mg cellulase plus b-glucosidase mass loading, respectively) was found between the increase in glucose release and the increase in xylose release. However, it is also interesting to note that the impact of xylanase was more sensitive to enzyme loadings for ARP than AFEX, which means that with increased cellulase loading the leverage factors for ARP decreased more than for AFEX. In any event, linear relationships of this nature were observed for enzymatic digestion with xylanase supplementation of solids prepared by the other pretreatments, inferring that enhancing xylan removal with xylanase made glucan more accessible to cellulase.

As shown in Table 3, xylanase leverage varied with the type of pretreatment. Furthermore, while ARP pretreated solids showed the highest xylanase leverage for a cellulase mass loading of 14.5 mg, controlled pH pretreated solids had the highest glucose release for a given increase in xylose release at a cellulase mass loading of 29.0 mg.

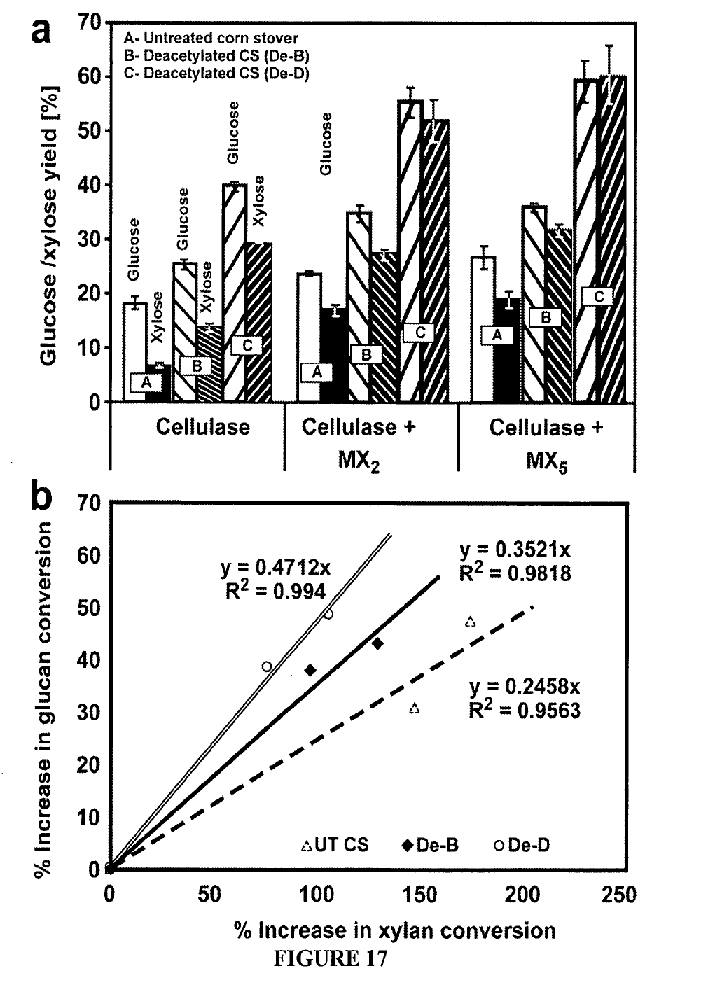

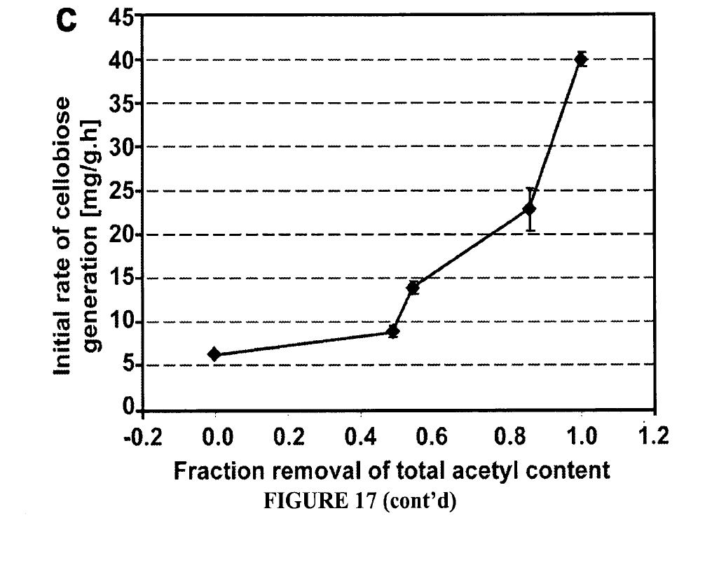

To determine the role of acetyl groups in xylanase leverage and whether their removal enhanced cellulose accessibility, two separate set of hydrolysis experiments were run for corn stover solids, which were deacetylated to various degrees, with cellulase plus bglucosidase supplemented with xylanase and with just purified CBH-I (Cel7A). As shown in FIG. 17a for a cellulase plus b-glucosidase mass loading of 29.0 mg with xylanase supplementation mass ratios of 2 and 5 (MX2 and MX5), xylanase supplementation increased glucose and xylose release much more for deacetylated corn stover (De-B and De-D) compared to untreated corn stover (UT CS). In addition, the linear relationships between increases in glucose and xylose release FIG. 17b suggest that leverage increased with removal of acetyl groups. For example, glucose release increased almost twice as much with xylose removal for completely deacetylated corn stover compared to untreated corn stover and about 1.3 times as much when 50% of the acetyl content was removed. As shown in FIG. 17c, deacetylation of corn stover enhanced cellobiose production by purified CBH-I at a loading of 15 mg/g glucan, with generation increasing by about 530% for complete removal. Hence, removal of acetyl groups not only increased xylanase leverage but enhanced cellulose accessibility and/or CBHI effectiveness as well. Yet, the fact that lime pretreated solids with the lowest residual acetyl groups did not display the highest xylanase leverage suggests that acetyl content is not the only factor controlling xylanase leverage. Furthermore, no clear correlation was found between acetyl content and the degree of xylanase leverage in this study.

To unravel this complex situation, xylanase leverage data were correlated with substrate features and enzyme loadings using the statistical software Minitab based on the xylanase leverage data; cellulase mass loadings; acetyl, xylan, and lignin content; and cellulose crystallinity index (CrI) summarized in Table 4. The multiple variables model used for linear regression is as follows:

Y=A₀+A₁*[CrI]+A₂*[% acetyl content]+A₃*[% glucan]+A₄*[% xylan]+A₅*[% lignin]+A₆%[cellulase loading]

in which Y=xylanase leverage, as defined by the ratio of percentage increase in glucan conversion to percentage increase in xylan conversion, A₀ to A₆=constants, and cellulase loading=mg protein/g original glucan.

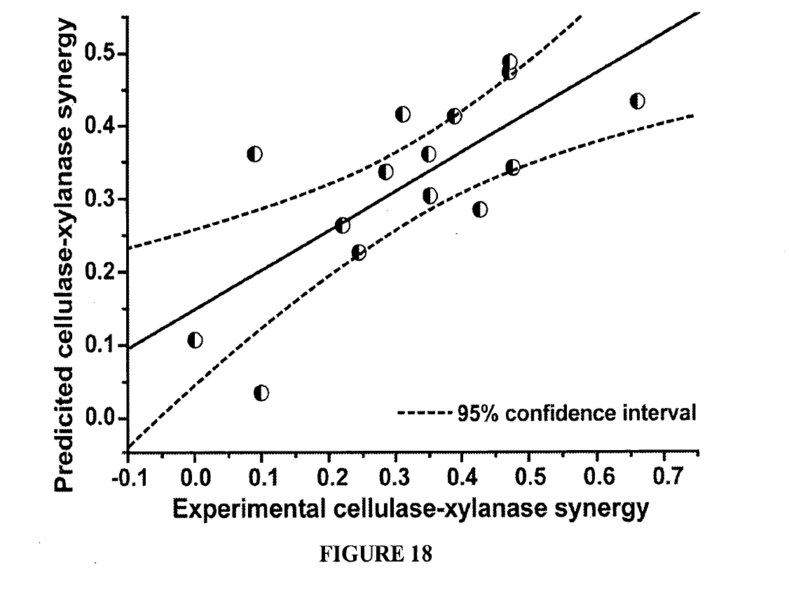

The parameters for substrate features and cellulase loadings were linearly regressed to give the coefficients shown in Table 5 for the best fit as determined in terms of the statistical coefficient R2 (0.537). The low value of the statistical coefficient R2 indicates that leverage is more complex than can be explained by this model, and further refinement is needed in the parameters included and their relationships to leverage. Nonetheless, although the correlation was not great, acetyl content appeared to have the biggest impact on xylanase leverage, followed by crystallinity of biomass and lignin content. However, the importance of the parameters may also be interpreted based on their p values (set value 0.1 at 90% confidence interval) shown in Table 5, from which crystallinity index would be concluded to have the greatest effect on xylanase leverage. Experimental and predicted values of xylanase leverage factors (LF) are shown in Table 4 and FIG. 18.

TABLE 4
Cellulase-xylanase leverage data and substrate features for unpretreated and deacetylated
corn stover and corn stover pretreated by leading pretreatment technologies.

| | | | | % Acetyl | | Cellulase | Xylanase leverage | Xylanase leverage |
|---|---|---|---|---|---|---|---|---|
| Pretreatment | % Glucan | % Xylan | % Lignin | content | Crl^{d} | loading^{e} | factor (exp.) | factor (model) |
| UT- CS | 38.3 | 21.7 | 20.5 | 2.51 | 50.3 | 29.0 | 0.2458 | 0.2262 |
| De-B^{a} | 43.0 | 26.4 | 19.7 | 1.15 | 50.3^{c} | 29.0 | 0.3521 | 0.3055 |
| De-D^{b} | 42.1 | 23.4 | 19.5 | 0.03 | 50.3^{c} | 29.0 | 0.4712 | 0.4763 |
| AFEX | 34.4 | 22.8 | 18.0 | 1.70 | 36.3 | 14.5 | 0.3898 | 0.4129 |
| ARP | 61.9 | 17.9 | 8.8 | 0.30 | 25.3 | 14.5 | 0.4741 | 0.3423 |
| DA | 59.3 | 9.3 | 22.5 | 1.13 | 52.5 | 14.5 | 0.2210 | 0.2645 |
| Lime | 56.7 | 26.4 | 14.6 | 0.30 | 56.2 | 14.5 | 0.0988 | 0.0333 |
| Controlled pH | 52.7 | 16.2 | 25.2 | 1.70 | 44.5 | 14.5 | 0.0892 | 0.3609 |
| SO₂ | 56.9 | 11.6 | 23.8 | 1.15 | 53.0 | 14.5 | 0.4274 | 0.2864 |
| AFEX | 34.4 | 22.8 | 18.0 | 1.70 | 36.3 | 29.0 | 0.4725 | 0.4862 |
| ARP | 61.9 | 17.9 | 8.8 | 0.30 | 25.3 | 29.0 | 0.3112 | 0.4156 |
| DA | 59.3 | 9.3 | 22.5 | 1.13 | 52.5 | 29.0 | 0.2859 | 0.3378 |
| Lime | 56.7 | 26.4 | 14.6 | 0.30 | 36.3 | 29.0 | 0.0009 | 0.1066 |
| Controlled pH | 52.7 | 16.2 | 25.2 | 1.70 | 44.5 | 29.0 | 0.6600 | 0.4342 |
| SO₂ | 56.9 | 11.6 | 23.8 | 1.15 | 53.0 | 29.0 | 0.3485 | 0.3597 |

^{a}~54% deacetylated corn stover.
^{b}~100% deacetylated corn stover.
^{c}It was assumed that with deacetylation the Crt of biomass does not change.
^{d}Crystallinity values were taken from Laureano-Perez et al. (2005).
^{e}Cellulase loading- mg/g glucan in unpretreated corn stover.

TABLE 5
Variable coefficients, their standard error, and significance (p value).

| Predictor | Coefficient | Standard error of coeff. | p value |
|---|---|---|---|
| Constant [A0] | 1.110 | 0.791 | 0.198 |
| Crl [A1] | −0.013 | 0.007 | 0.087 |
| % Acetyl content [A2] | −0.128 | 0.097 | 0.225 |
| % Glucan [A3] | −0.008 | 0.008 | 0.350 |
| % Xylan [A4] | −0.008 | 0.014 | 0.597 |
| % Lignin [A5] | 0.022 | 0.019 | 0.275 |
| Cellulase loading [A6] | 0.005 | 0.006 | 0.416 |

Several studies reported a strong relationship between xylan removal and the extent of glucan digestion and two hypothesized mechanisms are removal of xylan that coats glucan chains, making them more accessible to cellulase, and disruption of xylan linkages to glucan. In the case of xylanase leverage, a plausible explanation could be that xylanase removes redeposited xylan and xylooligomers from the surface, thereby increasing the accessibility of cellulose microfibrils to cellulase. However, the extent of xylan and/or xylooligomers aggregation on cellulose and the strength of its binding by covalent and hydrogen bonds are affected by physical features, the type of xylan, cellulose characteristics, and the presence of lignin In addition, xylan accessibility could in turn be limited by the presence of glucan microfibrils as xylan is believed to be intertwined with glucan chains and may play a vital role in xylanase leverage on cellulase action.

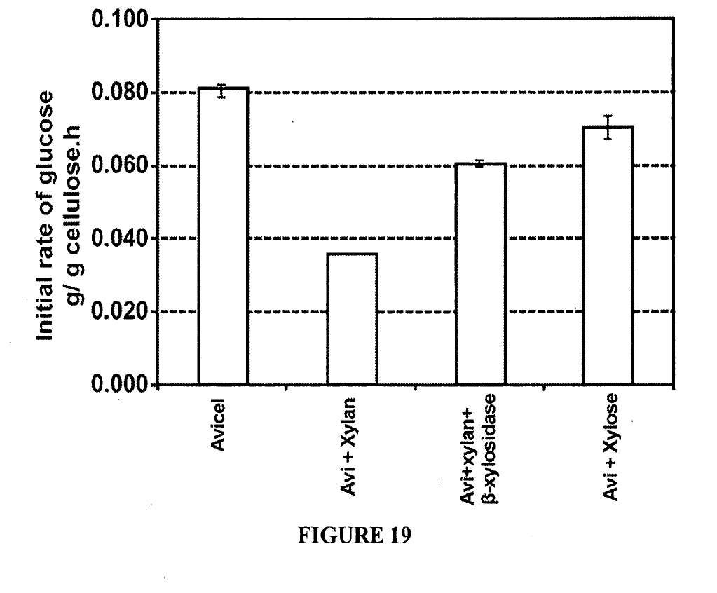

Sugars and oligomers released during hydrolysis could also be factors. The low xylanase activity in commercial enzymes produces a significant amount of xylooligomers during enzymatic hydrolysis of biomass solids that contain xylan, and these xylooligomers are strongly inhibitory to cellulase. For example as shown in FIG. 19, the initial rate of glucose release during hydrolysis of Avicel glucan mixed with an equal amount of birchwood xylan at a cellulase loading of 7.5 FPU/g glucan dropped by 55% compared to hydrolysis of Avicel alone, While xylose mixed with Avicel in an amount equivalent of xylan, had a limited inhibition of enzyme action (12%), inhibition by xylose, xylooligomers, and/or xylan was reduced by ˜70% when b-xylosidase was added to the cellulase.

Thus, removing xylooligomers by b-xylosidase or its activity in xylanase reduces inhibition of cellulase and speed conversion.

Other features and advantages of the disclosure will become more apparent from the following detailed description of the invention, when taken in conjunction with the accompanying exemplary drawings. Further modifications and improvements may additionally be made to the system and methods disclosed herein without departing from the scope of the disclosure. Accordingly, it is not intended that the invention be limited by the embodiments disclosed herein.

We claim:

1. A method for digesting a lignocellulosic biomass, comprising:
treating a lignocellulosic biomass with one or more enzymes that remove oligomers of xylose and a cellulase.

2. The method for digesting a lignocellulosic biomass of claim 1, wherein the enzyme is chosen from the group consisting of β-xylosidase, xylanase, and other hemicellulase enzymes that breakdown oligomers of xylose.

3. The method for digesting a lignocellulosic biomass of claim 1, further including subjecting the lignocellulosic biomass to a pretreatment process, wherein the pretreatment process is chosen from the group consisting of ammonia fiber expansion (AFEX), ammonia recycle (ARP), adding dilute sulfuric acid, adding lime, processing under neutral pH conditions, and treatment with sulfur dioxide.

4. The method for digesting a lignocellulosic biomass of claim 1, wherein the lignocellulosic biomass includes the solids and liquids or just the solids resulting from pretreatment of cellulosic biomass chosen from the group consisting of corn stover, switchgrass, birch wood, poplar wood, Miscanthus wood and aspen wood.

5. The method of claim 1, wherein the enzyme that remove oligomers of xylose is contacted with the lignocellulosic biomass prior to contact with a cellulase.

6. The method of claim 1, wherein the enzyme that remove oligomers of xylose is contacted with the lignocellulosic biomass prior to treatment with a pretreatment process selected from the group consisting of ammonia fiber expansion (AFEX), ammonia recycle (ARP), adding dilute sulfuric acid, adding lime, processing under neutral pH conditions, and treatment with sulfur dioxide.

7. A method for digesting a lignocellulosic biomass, comprising:
treating a lignocellulosic biomass with one or more enzymes that remove xylooligomers and higher chain length oligomers of glucose; and
processing the enzyme treated lignocellulosic biomass with a cellulase and/or xylanase.

8. The method for digesting a lignocellulosic biomass of claim 7, wherein the enzyme is chosen from the group consisting of β-xylosidase, xylanase, and other hemicellulase enzymes that breakdown oligomers of xylose.

9. The method for digesting the solids or the solids and the liquid from lignocellulosic biomass of claim 7, produced by a pretreatment process, wherein the pretreatment process is chosen from the group consisting of ammonia fiber expansion (AFEX), ammonia recycle (ARP), adding dilute sulfuric acid, adding lime, processing under neutral pH conditions, and treatment with sulfur dioxide.

10. The method for digesting a lignocellulosic biomass of claim 7, wherein the lignocellulosic biomass includes the solids and/or the liquid resulting from pretreatment of cellulosic biomass chosen from the group consisting of pretreated corn stover, pretreated poplar wood, pretreated switchgrass, pretreated Miscanthus, pretreated aspen, and many other types of agricultural and forestry residues, municipal solid waste, grasses, and woody plants.

11. The method of claim 7, wherein the enzyme that remove oligomers of xylose is contacted with the lignocellulosic biomass prior to contact with a cellulase.

12. The method of claim 7, wherein the enzyme that remove oligomers of xylose is contacted with the lignocellulosic biomass prior to treatment with a pretreatment process selected from the group consisting of ammonia fiber expansion (AFEX), ammonia recycle (ARP), adding dilute sulfuric acid, adding lime, processing under neutral pH conditions, and treatment with sulfur dioxide.

13. A composition, comprising:
a lignocellulosic biomass;
an enzyme chosen from the group consisting of β-xylosidase and xylanase; and
a cellulase enzyme.

14. A reactor system for producing a biofuel comprising a lignocellulosic biomass, an enzyme that degrades xylose or xylose oligomers and a cellulase.

15. The reactor system of claim 14, further comprising an acetyl neutralizing or removal system.
